(19) United States
(12) Patent Application Publication
(10) Pub. No.: US 2004/0076984 A1
(43) Pub. Date: Apr. 22, 2004

(54) Expert system for classification and prediction of generic diseases, and for association of molecular genetic parameters with clinical parameters

(76) Inventor: Roland Eils, Mannheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/433,840

(22) PCT Filed: Dec. 7, 2001
(86) PCT No.: PCT/EP01/14407

(30) Foreign Application Priority Data
Dec. 7, 2000 (EP) 00126480.3

(51) Int. Cl. C12Q 1/68; G06F 19/00; G01N 33/48; G01N 33/50

(52) U.S. Cl. 435/6; 702/20

(57) ABSTRACT

The present invention is directed to methods, devices and systems for classifying genetic conditions, diseases, tumors etc., and/or for predicting genetic diseases, and/or for associating molecular genetic parameters with clinical parameters and/or for identifying tumors by gene expression profiles, etc. The invention specifies such methods, devices and systems with the steps of providing molecular genetic data and/or clinical data, automatically classification, prediction, association and/or identification data by means of a supervising machine learning system. There are further described methods making use of these steps and respective means.

This invention relates to a proprietary expert system, in particular a data mining system, for classification and prediction of genetic diseases according to clinical and/or molecular genetic parameters. The invention more particularly relates to a decision support or assist system which is particularly adapted to assist the clinician in assessment of prognosis and therapy recommendation. Furthermore, this system allows the association of clinical parameters such as survival, diagnosis and therapy response with molecular genetic parameters. The data mug system consists of machine learning approaches (artificial neural networks, decision tree/rule induction method, Bayesian Belief Networks) and several different clustering approaches.

Classification of human tumors into distinguishable entities is preferentially based on clinical, pathohistological, enzyme-based histochemical, immunohistochemical, and in some cases cytogenetic data. This classification system still provides classes containing tumors that show similarities but differ strongly in important aspects, e.g. clinical course, treatment response, or survival. Thus, information obtained by new techniques like cDNA microarrays that are profiling gene expression in tissues might be beneficial for this dilemma.

The identification of relevant information with biological importance has come to a new age with emerging technologies that provide the research community with vast amounts of data at comparatively short experimental time costs. Array approaches like cDNA, RNA, and protein chips accumulate information regarding gene expression levels and protein status, respectively, of different tissues including those of tumor origin that can hardly be investigated with standard biostatistical methods.

The analysis of gene microarray data is hampered by its characteristic complexity. In general, a typical data set is described by a n×m matrix of n patients and m gene expression levels. Typically, m is larger than n by a factor of 10 to 100, and the characterizing features are real number values.

Without appropriate statistical tools significant perceptions bidden in the pool of data might not be recognized. Therefore, methods capable of handling large data sets of thousands of attributes are demanded.

EP 1 037 158 A2 relates to methods and an apparatus for analyzing gene expression data, in particular for grouping or clustering gene expression patterns from a plurality of genes. This prior art utilizes a self organizing map to cluster the gene expression patterns into groups that exhibit similar patterns.

EP 1 043 676 A2 relates to methods for classifying samples and ascertaining previously unknown classes. There is disclosed a method for identifying a set of informative genes whose expression correlates with a class distinction between samples with the steps of sorting genes by degree to which their expression in the samples correlate with a class distinction and determining whether the correlation is stronger than expected by cue. More particularly, a method is described for assigning a sample to a known or putative class by a weighted voting scheme.

It is the object underlying the present invention to provide a method, a computer programs and a computer system for classifying genetic diseases, tumors etc., and/or for predicting genetical diseases, and/or for associating molecular genetic parameters with clinical parameters and/or for identifying tumors by gene expression profiles etc. It is also an object co provide data, genes or genetic targets obtainable by a method, a computer programm and a computer system according to the present invention and further methods and devices malting use of the above mentioned methods.

These objects are achieved with the subject-matter as recited in the claims and in the description.

The present invention relates to a method and system for classifying genetic conditions, diseases, tumors etc., and/or for predicting genetic diseases, and/or for associating molecular genetic parameters with clinical parameters and/or for identifying tumors by gene expression profiles etc., with the following features: providing molecular genetic data and/or clinical data, optionally automatically generating classification, prediction, association and/or identification data by means of machine learning, and automatically generating (further) classification, prediction, association and/or identification data by means of supervised machine learning. The use of the supervised machine learning according to the present invention leads to surprisingly better and more reliable results.

Preferably molecular genetic data and clinical data are provided.

Further preferably the machine learning system is an artificial neural network learning system (ANN), a decision tree/rule induction system and/or a Bayesian Belief Network.

Further preferably for generating the data in the machine learning system at least one decision tree/rule induction algorithm is used.

Further preferably, the data automatically generated is tumor identification data making use of gene expression profiles and being generated by a clustering system wherein further the clustering system makes use of one or more of the following clustering methods: Fuzzy Kohonn Networks, Growing cell structures (GCS), K-means clustering and/or Fuzzy c-means clustering.

Further preferably, the data automatically generated is tumor classification data being generated by Rough Set Theory and/or Boolean reasoning.

Further preferably, for automatically generating the data use is made of FISH, CGH and/or gene mutation analysis techniques.

Further preferably, data is collected by means of gene expression techniques, preferably by cDNA microarrays, and then analyzed for providing the molecular genetic data.

The preset invention is also directed to a computer program comprising program code means for performing the method of any one of the preceding embodiments when the program is run on a computer. Further preferably, the computer program product comprises program code means stored on a computer readable medium for performing the above mentioned method when said program product is run on a computer.

The invention also concerns a computer system, particularly for performing the above method with means for providing molecular genetic data and/or clinical data, optional means for automatically generating classification, prediction, association and/or identification data by means of a machine learning system, and means for automatically generating (further) classification, prediction, association and/or identification data by means of a supervising machine learning systems. This system can be provided in the form of an expert system and/or classification systems with the help of symbolic and subsymbolic machine learning approaches. Such a system can assist the clinician in the assessment of the prognosis and/or therapy recommendation.

The invention also embraces a method for the production of a diagnostic composition comprising the steps of the above method and the further step of preparing a diagnostically effective device and/or collection of genes based on the results obtained by the above method.

Further, the invention also embraces the use of a gene or a collection of genes for the preparation of a diagnostic composition for classifying genetic diseases, tumors etc., and/or for predicting genetic diseases, and/or for associating molecular genetic parameters with clinical parameters and/or for identifying tumors by gene expression profiles etc.

The invention relates in addition to a method for determining a treatment plan for an individual having a disease, such as cancer, with the following steps: obtaining a sample from the individual, deriving individual molecular genetic data and/or clinical data from the sample, using the above classifying method, comparing the individual molecular genetic data and/or clinical data from the sample with the classification obtained by tee classifying method and determining a treatment plan according to the classification result.

The present invention is also directed to a method for diagnosing or aiding in the diagnosis of an individual with the following steps: obtaining a sample from the individual, deriving individual molecular genetic data and/or clinical data from the sample, using the above classifying method, comparing the individual molecular genetic data and/or clinical data from the sample with the classification obtained by the classifying method, determining a treatment plan according to the classification result and diagnosing or aiding in the diagnosis of the individual.

The invention relates also to a method for determining a drug target of a condition or disease of interest with the following steps: obtaining a classification with the above method and determining genes that are relevant for the classification of a class.

Even further, the invention concerns a method for determining the efficiency of a drug designed to treat a disease class with the following steps: obtaining a sample from an individual having the disease class, subjecting the sample to the drug, classifying the drug exposed sample with the above method.

The method according to the present invention can also be used for determining the phenotypic class of an individual with the following steps: obtaining a sample from the individual, deriving individual molecular genetic data and/or clinical data from the sample, establishing a model for determining the phenotypic classes with the above method, and comparing the individual data with the model.

The person skilled in the art will appreciate that there are other applications for the invention and the above described methods and systems. The invention and particularly preferred embodiments thereof will be further explained below.

Preferred Molecular Classification of Cancer and Gene Identification by Symbolic and Subsymbolic Machine Learning Approaches

Based on microarray gene expression, The invention is directed to two machine learning techniques in the context of molecular classification of cancer and identification of potentially relevant genes. The techniques in question are (1) decision trees (symbolic approach) and (2) artificial neural networks (subsymbolic approach). Commonly, decision trees are said to be advantageous in situations where the complexity is relatively low (small number of variables and low degree of interrelation among variables) and the variables are directly interpretable by human (numeric variables such as Age, Cholesterol, etc., and symbolic variables such as Gender, tumor stage etc.). Artificial neural networks on the other hand are preferable embodiments in situations where there are many interacting variables (e.g., images) and non-linear behavior of the underlying phenomena.

As a basis for a comparative study two of the most popular algorithms currently available in machine learning software were chosen, namely the decision tree/rule induction algorithms C5.0 and the backpropagation algorithm for multilayer perceptrons (MLP), a specific architecture of artificial neural networks (ANN) [2,3,4]. For both algorithms we used the proprietary implementation realized in the data mining tool Clementine from SPSS [5].

The general approach was to directly use (as provided on the Web) all expression data (except the control data) without further processing, and

1. to determine, compare, and explain (factors that lead to the classification results) the classification performance of both methods based on n-fold cross-validation procedure and the lift measure [3] commonly used by the machine learning community. We have randomly subsampled the entire set of n=72 cases into five training sets (n₁=15) and five test sets (n₂=57), plus the original gaining data set (n₁=38) and test set (n₂=34) supplied on the Web.

2. to analyze the entire set of 72 cases and determine the genes that are most relevant for the classification of the underlying tumor classes.

Summary of Results:

ANN Classification:

Each MLP was composed of one input, two hidden and one output layer. The most complex architecture consisted of six nodes in the first and four nodes in the second hidden layer. The least complex architecture consisted of two nodes in the first and two nodes in the second hidden layer. The neurons in the hidden layers were pruned and generated dynamically. Training times for each neural network model was limited to a maximum of 5 minutes.

The best classification performance was obtained by interrupting the learning process between 85% and 90% (average: 88.43%) predicted accuracy. In this case the average classification accuracy over all 6 cross-validation runs was 84.35%.

Training the net to a predicted accuracy, x, of x>90% and 80%<x<85%, respectively, resulted in lower actual prediction performances (namely 78.79% in the former and 71.77% in the latter case).

Further analysis showed that although for each of the three neural net rules the ALL tumor was classified with a higher accuracy than the AML class: ALL avg. classification accuracy over all three runs. 92.76%, for AML: 54.74%. However, the lift measure for the AML class scored higher in each of the test runs: ALL avg. lift score over all three runs: 1.52, for AML: 2.04. This means that the model showed a definitely higher sensitivity/selectivity with regard to the AML class. See also Table 1 for a summary of these results.

C5.0 Decision Tree Classification:

The best classification performance of the C5.0 decision tree method was obtained on the basis of 20 fold boosting (combination of multiple definitely different models). In this case the average classification accuracy over all 6 cross-validation runs was 92.98%. The result for 10 fold boosting was only marginally lower (91.87%). However, the non-boosting version of the decision tree only achieved an average classification accuracy of 84.09%. Interestingly, for the common training set (n=38) provided for the competition, the boosting method was not able to derive multiple models, but repeated the known result: Zyxin (accession code X95735_at) with an expression level of 938 as decision boundary. However, for many of The other cross-validation subsamples, boosting was able to identify multiple complementary models, thus indicating multiple genes and expression levels related to differentiating AML and ALL. A list of these genes will be provided.

Further analysis showed that over all three C5.0 decision tree runs the AML class was classified with a higher accuracy than ALL. Avg. classification accuracy over all three runs: 90.94% for AML, and 88.28% for ALL. Moreover, the lift measure for the AML class scored significantly higher in each of the three test runs (ALL avg. lift score over all Bee runs; 1.50, for AML: 2.44). This means that the C5.0 decision tree model not only showed a significantly higher sensitivity/selectivity with regard to the AML class (when compared with ALL), but also a slightly higher precision. See also Table 1 for a summary of these results. With regard to the ALL class both models showed comparable results regarding lift (sensitivity/selectivity) and precision (accuracy), but for the AML the decision tree method clearly outperformed the neural net approach.

Training times the C5.0 decision tree model construction ranged from 10-20 seconds for the non-boosting to 10-30 seconds for 10 fold boosting to 100 seconds for 20 fold boosting.

TABLE 1
Summary of results.

| | Neural Network | | | | | | Decision Tree | | | | | |
|---|---|---|---|---|---|---|---|---|---|---|---|---|
| | Accuracy % | | | Lift | | | Accuracy % | | | Lift | | |
| Test Set(s) | AML | ALL | TOT | AML | ALL | TOT | AML | ALL | TOT | AML | ALL | TOT |
| Best | 50.00 | 100.0 | 79.41 | 2.42 | 1.25 | 1.84 | 92.85 | 90.00 | 91.18 | 2.10 | 1.61 | 1.85 |
| Competition Set | | | | | | | | | | | | |
| Best | 67.08 | 94.63 | 84.35 | 2.61 | 1.55 | 1.98 | 92.56 | 92.62 | 92.97 | 2.64 | 1.54 | 2.09 |
| Performance | | | | | | | | | | | | |
| Run | | | | | | | | | | | | |
| Best Single Test | 75.00 | 100.0 | 93.33 | 3.74 | 1.25 | 2.50 | 100.0 | 100.0 | 100.0 | 3.74 | 1.36 | 2.56 |
| Set | | | | | | | | | | | | |
| All 3 Test Runs | 54.74 | 92.76 | 78.30 | 2.04 | 1.52 | 1.66 | 90.94 | 88.28 | 89.64 | 2.44 | 1.36 | 2.56 |

Gene Identification:

A list of the fifty most relevant genes based on all 72 cases was generated through boosting (C5.0) and sensitivity analysis (back-propagation). The sensitivity analysis for ranking and identifying high-impact variables was found easier to use, as it provided a direct ranking of the genes.

The comparison of the two methods shows that (1) Both can be used directly (no further preprocessing or discretization) with high dimensional inputs (>7000 genes) for molecular tumor classification and gene identification, (2) the C5.0 decision tree seems to be the preferred classification model as it (a) showed higher precision and sensitivity levels, (b) provides an output format that is easy to interpret by humans (symbolic rules), and (c) was faster to train than the neural model. It must be said however, that in the presence of more cases, the neural model may become more important (performant). Also, sensitivity analysis for ranking and identifying high-impact variables was found easier to use, as it provided a direct rig of the genes.

### REFERENCES

[1] Golub T R, Slonim D K, Tanayo P, Huard C, Gaasenbeek M, Mesirov J P, Coller A, Loh M L, Downing J R, Caligiuri M A, Bloomfield C D, Lander E S. Molecular classification of cancer: class discovery and class prediction by gene expression monitoring. Science 286(5439): 531-537, 1999.

[2] Werbos, P. J.: Beyond Regression, Doctoral Dissertation, Appl. Math., Harvard University, November 1974.

[3] Rumelhart, D. E. et al.: Parallel Distributed Processing, Vol. 1, MIT Press Cambridge, 1986.

[4] J. E. Dayhoff, “Neural Network Architectures: An Introduction”, Thomson Computer Press, 1996.

[5] SPSS: http://www.spss.com/datamine/, and Clementine User Group: http://www.spss.com/clementine/clug/

Tumor Identification by Gene Expression Profiles Using Five Different Clustering Methods

Tumors are generally classified by means of classical parameters such as clinical course, morphology and pathohistological characteristics. Nevertheless, the classification criteria obtained with these methods are not sufficient in every case. For example, it creates classes of cancer with significantly differing clinical courses or treatment response. As advanced molecular techniques are being established, more information about tumors is accumulated. One of these techniques, cDNA microarray, is profiling the expression of up to many thousand genes in one single experiment of a tissue sample, e.g. a tumor. The derived data may contribute to a more precise tumor classification, identification or discovery of new tumor subgroups, and prediction of clinical parameters such as prognosis or therapy response.

Clustering techniques are often used when there is no class to be predicted or classified but rather when cases are to be divided into natural groups. Clustering is concerned with identifying interesting patterns in a data set and describing them in a concise and meaningful manner. More specifically, clustering is a process or task that is concerned with assigning class membership to observations, but also with the definition or description of the classes that are used. Because of this added requirement and complexity, clustering is considered a higher-level process than classification. In general, clustering methods attempt to produce cases that maximize similarity within classes but minimize similarity between classes. In the context of microarray data analysis, clustering methods may be useful in automatically detecting new subgroups (e.g., tumors) in the data

The gene expression profiles of 72 patients diagnosed as either acute myeloid leukemia (AML) or acute lymphatic leukemia (ALL) [1] were taken to compare five clustering methods in respect of their ability to automatically partition this data set in clusters of corresponding cases. In this study, five clustering methods have been applied to the expression data (except controls):

1. Kohonen network; Kohonen networks or self-organizing feature maps (SOFMs) define a mapping from an n-dimensional input data space onto a one- or two-dimensional array of nodes [2]. The mapping is performed in a way that the topological relationships in the input space are maintained when mapped to the network grid (also called feature map). Furthermore, local density of data is also reflected by the map, that is areas of the input data space which are represented by more data are mapped to a larger area on the feature map. The basic learning process in a Kohonen network is defined as follows. (1) Initialize net with n nodes; (2) Select a case from the set of training cases; (3) Find node in net that is closest (according to some measure of distance) to the selected case; (4) Adjust the set of weight weights of the closest node and nodes around it; and (5) Repeat from step (1) until some termination criteria is reached. The amount of adjustment in step (4) as well as the range of the neighborhood decreases during the training. So coarse adjustments occur in the first phase of the training, while fine tuning occurs towards the end. Some of the issues in Kohonen learning are the settings for the learning parameters that determine the adjustments in step (4).

2. Fuzzy Kohonen networks: A fuzzy Kohonen networks combine concepts of fizzy set theory and standard SOFMs. The two major parts of fuzzy Kohonen networks are Kohonen networks and the fuzzy c-means clustering algorithm. The use of both techniques in one model aims at synthesizing the advantages of the two approaches to overcome some of the shortcomings of each individual technique such as the Kohonen learning parameter setting outlined above [3,4]. The Fuzzy Kohonen networks approach constitutes the most preferred embodiment of the invention in this context.

3. Growing cell structures (GCS): GCS neural networks constitute a generalization of the Kohonen network or SOFM approach. GCS offers several advantages over both non-self-organizing neural networks and self-organizing Kohonen networks [5]. Sole of those advantages are: (1) GCS is a neural network with a self-adaptive topology which is highly independent of the user; (2) the GCS self-organizing model consists of a small number of constant parameters; there is no need to define time-dependent or decay schedule parameters (the critical learning parameters of the standard Kohonen networks); and (3) the ability GCS to interrupt and resume the learning process permits the constructions of incremental and dynamic learning systems.

4. K-means clustering [6]: A classical representative of clustering methods is the k-means algorithm. This simple algorithm is initialized with the number of clusters being sought (the parameter k). Then: (1) k points are chosen at random as cluster centroids or centres; (2) the cases are assigned to the clusters by finding the nearest centroid) (3) Next new centroids of the clusters are calculated by averaging the positions of each point in the cluster along each dimension moving the position of each centroid; and (4) this process is repeated from step (2) until the boundaries of the clusters stop changing. One problem of the standard k-means is that the clustering result is heavily dependent on the selection of the initial seeds. The classical representative of clustering methods is the k-means algorithm. This simple algorithm is initialized with the number of clusters being sought (the parameter k). Then, in its simple standard implementation (1) k points are chosen at random as cluster centroids; (2) the cases are assigned to the clusters by finding the nearest centroid; (3) Next new centroids of the clusters are calculated by averaging the positions of each point in the cluster along each dimension moving the position of each centroid; and (4) this process is repeated from step (2) until the boundaries of the clusters stop changing.

5. Fuzzy c-means clustering: Many classical clustering techniques assign an object or case to exactly one cluster (all-or-nothing membership) [7]. In some situations this may be an oversimplification, because often objects can be partially assigned into two or more classes. The fuzzy c-means clustering algorithm is based on this idea. Simply speaking, fuzzy c-means may be viewed as an attempt to overcome the problem of pattern recognition in the context of imprecisely defined categories [8]. Given n of cases and a number of classes, k, a main feature of the fuzzy c-means approach is that each object in the discerned set of objects is assigned k membership degrees, one for each of the k clusters under consideration. Thus, an object may be assigned to a set of categories with a varying degree of membership.

In this comparison it was aimed at comparing the characteristics of the five clustering methods in the context of the following analysis tasks:

reproduction/verification of the tumor classification given in the data set, i.e., AML and ALL;

discovery of novel subclasses within the given groups; and

discovery of associations/correlations between therapy response and gene expression patterns.

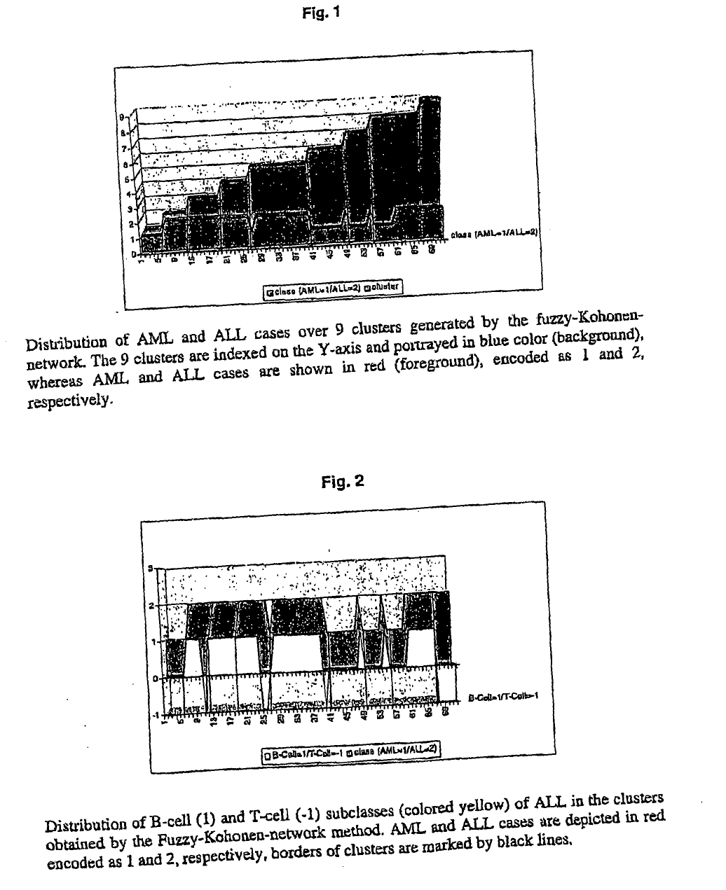

The five clustering methods produced between 2 and 16 clusters. The fuzzy Kohonen network was best at dividing the data set according to the respective gene expression profiles into clusters corresponding to biological classes. Best matches concerning the two classes AML and ALL was obtained by partitioning the set of all 72 cases into 9 clusters (cf. FIG. 1). Here, 5 clusters contained only ALL cases, one only AML cases, and within the remaining clusters there was only a single mismatch (either AML or ALL).

(see FIGS. 1 and 2)

Concerning subclasses of ALL (B-cell or T-cell ALL) fuzzy-kohonen was able to generate 3 clusters of either B-cell ALL or T-cell ALL, in 4 clusters only one case mismatched, in the remaining there were 2 cases not corresponding (cf. FIG. 2), Further subclasses of the groups were not found. Due to the small number of cases with treatment response data, none of the methods succeeded in clustering patients with similar treatment response. A comparison of the methods and the number of cases per cluster is given in table 1a (4 clusters generated) and 1b (6 clusters generated). Remarkably, k-means algorithm partitioned the data set considerably different when divided into 4 clusters, as did the kohonen network method as 6 clusters were demanded (only 3 clusters were generated).

TABLE 1
The number of cases per cluster of 4 clustering methods is demonstrated (a)
for performing 4 and (b) for 6 clusters.

| | Cluster | Cluster | | | | | | | | | |
|---|---|---|---|---|---|---|---|---|---|---|---|
| Table 1a | 1 | 2 | Cluster 3 | Cluster 4 | Table 1b | Cluster 1 | Cluster 2 | Cluster 3 | Cluster 4 | Cluster 5 | Cluster 6 |
| Fuzzy | 7 | 20 | 32 | 13 | Kohonen | 32 | 12 | 28 | — | — | — |
| Kohonen | | | | | | | | | | | |
| GCS | 14 | 22 | 19 | 17 | Fuzzy | 17 | 6 | 21 | 8 | 12 | 8 |
| | | | | | Kohonen | | | | | | |
| k-means | 46 | 1 | 2 | 23 | GCS | 14 | 15 | 9 | 12 | 12 | 10 |
| Fuzzy | 27 | 13 | 19 | 13 | Fuzzy | 12 | 18 | 7 | 10 | 12 | 8 |
| c-means | | | | | c-means | | | | | | |

Comparing five clustering methods in the context of realistic biological data resulted in one method to be the clear winner. The fuzzy Kohonen network provided a highly accurate and coherent division of the data set into corresponding groups or classes. After clustering the next step would be to identify the genes responsible for the clustering results (for example by applying classification methods to the most coherent cluster), and thus infer dependencies between highly predictive genes and the associated molecular genetic pathways,

### REFERENCES

[1] Golub T R, Slonim D K, Tamayo P, Huard C, Gaasenbeek M, Mesirov J P, Coller H, Loh M L, Downing J R, Caligiuri M A, Bloomfield C D, Lander E S. Molecular classification of cancer: class discovery and class prediction by gene expression monitoring. Science 286(5439): 531-537, 1999.

[2] Teuvo Kohonea: Self-Organizing Maps. Springer-Verlag, Heidelberg 1995

[3] Huntsberger T L and Aijimarangsee P. Parallel self-organising feature maps for unsupervised pattern recognition. In: Bezdek J. C, and Pal N. R, Editors: Fuzzy models for pattern recognition, pp 483-495. IEEE Press, New York, 1992.

[4] DataEngine. Manuals of the DataEngine software used in this analysis. MIT—Management Intelligenter Technologien GmbH. Aachen, Germany

[5] B. Fritzke, “Growing Cell Structures—A Self-Organizing Network for Unsupervised an Supervised Learning”, Neural Networks, vol. 7, pp. 1441-1460, 1994.

[6] Berry M J A, and Linoff G, Data mining techniques. For marketing, sales, and customer support. Wiley & Sons, Inc., 1997

[7] Anderberg M R. Cluster analysis for applications. Academic Press, New York; San Francisco, London, 1973.

[8] Bezdek J C. Pattern recognition with fuzzy objective function algorithms. Plenum Press, New York, London, 1981.

Preferred Embodiment for Mining Gene Expression Data using Rough Set Theory

Classification of human tumors into distinguishable entities is traditionally based on clinical, pathohistological, immunohistochemical and cytogenetic data. This classification technique provides classes containing tumors that show similarities but differ strongly in important aspects, e.g. clinical course, treatment response, or survival. New techniques like cDNA microarrays have opened the way to a more accurate stratification of patients with respect to treatment response or survival prognosis, however, reports of correlation between clinical parameters and patient specific gene expression patterns have been extremely rare. One of the reasons is that the adaptation of machine learning approaches to pattern classification, rule induction and detection of internal dependencies within large scale gene expression data is still a formidable challenge for the computer science community.

A preferred technique is applied based on rough set theory and Boolean reasoning [1,2] implemented in the Rosetta software tool [6]. This technique has already been successfully used to extract descriptive and minimal ‘if-then’ rules for relating prognostic or diagnostic parameters with particular conditions. The basis of rough set theory is the indiscernibility relation describing the fact that some objects of the universe are not discerned in view of the information accessible about them just forming a class. Rough set theory deals with the approximation of such sets of objects—the lower and upper approximations. The lower approximation consists of objects which definitely belong to the class and X upper approximation contains objects which possibly belong to the class, The difference between the upper and lower approximations—boundary region—consists of objects which cannot be properly classified by employing the available information.

The rough sets approach operates with data presented in a table called ‘decision table’ with rows corresponding to objects and columns corresponding to different attributes (‘condition attributes’). The data in the table is the result of evaluation of a given attribute on a given object. There is also a ‘decision attribute’ in the table, its values are the classes assigned to every object by an expert (‘decision classes’). The question is to what extent it is possible to infer from the condition attributes the classification carried out by an expert.

In this study, objects were the patients with two diseases: acute myeloid leukemia (AML) and acute lymphoblastic leukemia (ALL) [3]. Thus we had two decision classes: AML and ALL. Attributes in the table correspond to genes and attribute values are the gene expression data. The goal was to discover the attributes—genes that allow to discern between objects from different decision classes, while the objects within each class must not be discerned.

The Boolean function reflecting this discernibility can be constructed:

F(a₁, . . . , aᵢⱼ)= [symbol] (νcᵢⱼ),

cij={a|a(xᵢ)≠a(xⱼ)} for i=1, . . . , k₁, j=1, . . . , k₂,

where a₁, . . . , aᵢⱼ—Boolean variables, corresponding to the attributes, xᵢ—objects of the first decision class, xⱼ—objects of the second decision class.

It was shown [1] that the constituents in the minimal disjunctive normal form of this function are the minimal attribute sets that preserve the discernibility of objects of different decision classes. This minimal attribute sets are called ‘reducts’. The reducts are preferably calculated with the Rosetta software tool.

In order to compare the numerically valued attributes it was necessary to discretize the domains of the attributes. We have used only two values to express the two features of attributes—underexpression and overexpression of genes, encoding underexpression with 0 and overexpression with 1. A simple encoding method is preferred: for each attribute (gene) values larger than the mean were coded with 1 and values smaller than the mean with 0. It must be emphasized that different discretization techniques could bring different results. So discretization is a very important issue while adapting the machine leaning methodologies to the analysis of gene expression data,

Based on the obtained reduct sets, a set of decision rules were derived with combinatorial patterns of attribute values on the left side of the rules and AML or ALL decision classes on the right.

The quality of each rule was estimated by an algorithm of Michalski ([4], [5]) that computes a single value for rule quality based on two rule quality measures: classification accuracy and completeness.

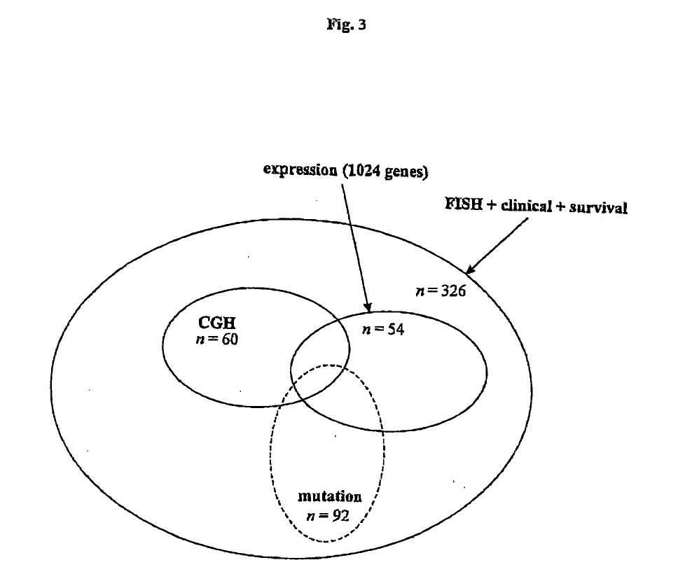

With the rough set theory approach described above, 1140 rules were obtained which were filtered with respect to their quality. 33 rules describing ALL cases and 19 rules for ALL remained after filtering. The most informative rules are presented in FIG. 1 and FIG. 2. The genes in the rules are denoted with g#, where # stands for the number of a gene in the training data set [3] (see the gene accession numbers and descriptions below), Furthermore, we have applied the rough sets methodology co derive the rules from the available information on therapy response of AML/ALL patients (see FIG. 3).

In conclusion, the application of rough set theory for mining gene expression data yields a large number of rules, which can be efficiently reduced to a smaller number of most significant rules by an automated approach.

FIG. 1.
Rules discriminating ALL class.

g895(0) AND g3096(0) AND g4848(0) => Class(ALL)
g93(1) AND g2001(1) => Class(ALL)
g93(1) AND g6364(0) => Class(ALL)
g93(1) AND g5694(1) => Class(ALL)
g2263(1) AND g6148(1) => Class(ALL)
g3709(0) AND g5269(0) AND g6148(1) => Class(ALL)
g679(1) AND g3048(0) => Class(ALL)
g1809(0) AND g3580(1) AND g3606(0) AND g7128(1) => Class(ALL)
g236(0) AND g962(1) AND g1809(0) AND g4187(1) AND g4815(1) =>
Class(ALL)
g4547(1) => Class(ALL)
g909(0) AND g1698(0) AND g5818(0) => Class(ALL)
g1698(0) AND g3794(0) AND g5818(0) => Class(ALL)
g578(1) AND g1698(0) AND g5818(0) => Class(ALL)
g1698(0) AND g3245(1) AND g5818(0) => Class(ALL)
g972(1) AND g2036(1) => Class(ALL)
g827(1) AND g6406(0) AND g7050(1) => Class(ALL)
g1134(0) AND g3868(1) AND g5050(1) => Class(ALL)
g737(1) AND g3172(1) AND g5688(1) => Class(ALL)
g5824(1) => Class(ALL)
g3255(1) AND g5570(1) => Class(ALL)
g3590(1) AND g5940(1) => Class(ALL)
g1129(0) AND g6627(1) => Class(ALL)
g1129(0) AND g6030(1) => Class(ALL)
g3596(1) AND g4510(1) AND g4685(1) => Class(ALL)
g243(0) AND g1129(0) AND g3596(1) => Class(ALL)
g995(1) AND g1633(1) AND g3674(1) AND g3853(0) AND g5869(1) =>
Class(ALL)
g3856(1) => Class(ALL)
g852(0) AND g5405(1) => Class(ALL)
g3830(1) AND g5632(1) => Class(ALL)
g3830(1) AND g5299(0) => Class(ALL)
g29(1) AND g3830(1) AND g4878(1) => Class(ALL)
g3830(1) AND g4834(1) AND g6025(1) => Class(ALL)

FIG. 2.
Rules discriminating AML class.

g2364(1) AND g3377(0) AND g3644(0) AND g3803(0) AND g4986(0) AND g5545(1) =>
Class(AML)
g3229(1) AND g3377(0) AND g3644(0) AND g3803(0) AND g4986(0) AND g5545(1) =>
Class(AML)
g2108(0) AND g2773(1) AND g3377(0) AND g3644(0) AND g3803(0) AND g4986(0)
AND g5545(1) => Class(AML)
g2108(0) AND g3377(0) AND g3644(0) AND g3803(0) AND g4986(0) AND g5545(1)
AND g5895(1) => Class(AML)
g3377(0) AND g3644(0) AND g3803(0) AND g4491(0) AND g4906(1) AND g4986(0)
AND g5545(1) => Class(AML)
g2108(0) AND g3377(0) AND g3644(0) AND g3803(0) AND g4083(1) AND g4986(0)
AND g5545(1) => Class(AML)
g2108(0) AND g3377(0) AND g3644(0) AND g3803(0) AND g4986(0) AND g5545(1)
AND g5754(0) => Class(AML)
g2108(0) AND g3377(0) AND g3644(0) AND g3803(0) AND g4770(1) AND g4986(0)
AND g5545(1) => Class(AML)
g1197(0) AND g1886(1) AND g3708(0) => Class(AML)
g506(0) AND g3009(1) AND g3044(0) AND g5224(0) AND g5864(0) AND g6444(1) =>
Class(AML)
g506(0) AND g608(1) AND g2995(0) AND g3044(0) AND g5224(0) AND g5864(0) AND
g6444(1) => Class(AML)
g506(0) AND g2995(0) AND g3044(0) AND g5224(0) AND g5864(0) AND g6444(1)
AND g6475(1) => Class(AML)
g506(0) AND g3009(1) AND g4095(0) AND g5224(0) AND g5864(0) AND g6444(1)
AND g6475(1) => Class(AML)

FIG 3.
Rules discriminating patients with successful treatment response.

g238(0) AND g1047(1) AND g1519(0) AND g2354(0) AND g2570(0)
AND g2951(1) AND g4070(1) AND g5495(0) AND g5914(1) AND
g6165(0) => Class(Success)
g238(0) AND g1047(1) AND g1519(0) AND g2354(0) AND g2570(0)
AND g2951(1) AND g4070(1) AND g4267(0) AND g5495(0) AND
g5914(1) => Class(Success)
g238(0) AND g1047(1) AND g1519(0) AND g2354(0) AND g2570(0)
AND g2951(1) AND g3028(0) AND g4070(1) AND g5495(0) AND
g6289(0) => Class(Success)
g238(0) AND g1047(1) AND g1519(0) AND g2354(0) AND g2570(0)
AND g2951(1) AND g3344(1) AND g4070(1) AND g5495(0) AND
g6841(1) => Class(Success)
g238(0) AND g1047(1) AND g1519(0) AND g2354(0) AND g2570(0)
AND g2951(1) AND g4070(1) AND g5495(0) AND g6165(0) AND
g6712(0) => Class(Success)

### REFERENCES

1. Z. Pawlak, Rough Sets—Theoretical Aspects of Reasoning about Data, Kluwer Academic Publishers, 1991

2. Ed. L. Polkowsky, Rough sets and current trends in computing, Proc. RSCTC '98, Warsaw, 1998

3. Golab T R, Slonim D K, Tamayo P, Huard C, Gaasenbeek M, Mesirov J P, Coller H, Loh M L, Downing J R, Caligiuri M A, Bloomfield C D, Lander E S. Science 286(5439): 531-537, 1999.

4. I. Bruha, Quality of Decision Rules: Definitions and Classifications, in Machine Leaning and Statistics, ed. G. Nakhaeizadeh, C. C. Tailor, 1999

5. T. Agotnes, J. Komorowski, A. Ohrn. Finding high performance subsets of induced rule sets: Extended summary, in Proc. Seventh European Congress on Intelligent Techniques and Soft Computing (EUFIT'99), Aachen, ed. H.-J. Zimmermann, K. Lieven, 1999

6. A. Ohrn, Discernibility and Rough Sets in Medicine: Tools and Application, Ph.D. Thesis

In the following, the gene identifiers are explained in further detail:

| Gene identifier | Gene Description | Gene Accession Number |
|---|---|---|
| 895 | Transcription Factor Iia | HG3162-HT3339_at |
| 3096 | GB DEF = Peroxisomal targeting signal import | U35407_at |
| | receptor (PXR1) gene, allele 5, partial cds | |
| 4848 | Zyxin | X95735_at |
| 93 | Cdc7-related kinase | AB003698_at |
| 2001 | GOT1 Glutamic-oxaloacetic transaminase 1, | M37400_at |
| | soluble (aspartate aminotransferase 1) | |
| 6364 | GB DEF = HOX7 gene, exon 2 and complete cds | M76732_s_at |
| 5694 | CCAAT transcription binding factor subunit | Z74792_s_at |
| | gamma | |
| 2263 | SMPD1 gene extracted from Homo sapiens acid | M81780_cds4_at |
| | sphingomyelinase (SMPD1) gene, ORF's 1-3′s | |
| 6148 | FMR2 Fragile X mental retardation 2 | X95463_s_at |
| 679 | KIAA0225 gene, partial cds | D86978_at |
| 3048 | Regulator of G-protein signaling similarity | U32439_at |
| | (RGS7) mRNA, partial cds | |
| 1809 | PDGFRA Platelet-derived growth factor receptor, | M21574_at |
| | alpha polypeptide | |
| 3580 | Kruppel-related zinc finger protein (ZNF184) | U66561_at |
| | mRNA, partial cds | |
| 3606 | Lysophospholipase homolog (HU-K5) mRNA | U67963_at |
| 7128 | RB1 Retinoblastoma 1 (including osteosarcoma) | L49218_f_at |
| 236 | KIAA0022 gene | D14664_at |
| 962 | Cpg-Enriched Dna, Clone S19 | HG3995-HT4265_at |
| 1809 | PDGFRA Platelet-derived growth factor receptor, | M21574_at |
| | alpha polypeptide | |
| 4187 | ANX8 Annexin VIII | X16662_at |
| 4815 | GB DEF = Ncx2 gene (exon 2) | X93017_at |
| 4547 | T-COMPLEX PROTEIN 1, GAMMA SUBUNIT | X74801_at |
| 909 | Thyroid Hormone Receptor, Beta-2 | HG3313-HT3490_at |
| 1698 | ERCC1 Excision repair cross-complementing | M13194_at |
| | rodent repair deficiency, complementation group | |
| | 1 (includes overlapping antisense sequence) | |
| 5818 | DNM1 Dynamin 1 | L07807_s_at |
| 3794 | Clone 23842 mRNA sequence | U79301_at |
| 578 | KIAA0170 gene | D79992_at |
| 3245 | GB DEF = G protein-coupled receptor GPR-9-6 | U45982_at |
| | gene | |
| 972 | Cytosolic Acetoacetyl-Coenzyme A Thiolase | HG4073-HT4343_at |
| 2036 | ME2 Malic enzyme 2, mitochondrial | M55905_at |
| 827 | Crystallin, Beta B3 (Gb:X15144) | HG2190-HT2260_at |
| 6406 | CD36 CD36 antigen (collagen type I receptor, | M98399_s_at |
| | thrombospondin receptor) | |
| 7050 | Chorionic somatomammotropin CS-1 gene | J03071_cds3_f_at |
| | extracted from Human growth hormone (GH-1 | |
| | and GH-2) and chorionic somatomammotropin | |
| | (CS-1, CS-2 and CS-5) genes | |
| 1134 | CATHEPSIN G PRECURSOR | J04990_at |
| 3868 | Lysyl hydroxylase isoform 2 (PLOD2) mRNA | U84573_at |
| 5050 | ANNEXIN XIII | Z11502_at |
| 737 | KIAA0276 gene, partial cds | D87466_at |
| 3172 | NAD(P) transhydrogenase | U40490_at |
| 5688 | SMCY (H-Y) mRNA | U52191_s_at |
| 5824 | PR264 gene | X75755_rna1_s_at |
| 3255 | Tetratricopeptide repeat protein (tpr1) mRNA | U46570_at |
| 5570 | Carboxyl Methyltransferase, Aspartate, Alt. | HG1400-HT1400_s_at |
| | Splice 1 | |
| 3590 | 3-hydroxyisobutyryl-coenzyme A hydrolase | U66669_at |
| | mRNA | |
| 5940 | Non-histone chromosomal protein HMG-14 | J02621_s_at |
| | mRNA | |
| 1129 | Alkaline phosphatase | J04948_at |
| 6627 | WSL-LR, WSL-S1 and WSL-S2 proteins | Y09392_s_at |
| 6030 | HISTATIN 3 PRECURSOR | M26665_at |
| 3596 | Multiple exostosis-like protein (EXTL) mRNA | U67191_at |
| 4510 | Variant hepatic nuclear factor 1 (vHNF1) | X71348_at |
| 4685 | FBLN2 Fibulin 2 | X82494_at |
| 243 | KIAA0110 gene | D14811_at |
| 1129 | Alkaline phosphatase | J04948_at |
| 3596 | Multiple exostosis-like protein (EXTL) mRNA | U67191_at |
| 995 | Cellular Retinol Binding Protein Ii | HG4310-HT4580_at |
| 1633 | Paraoxonase (PON2) mRNA | L48513_at |
| 3674 | H_LUCA 14.3 gene extracted from Human | U73167_cds4_at |
| | cosmid LUCA14 | |
| 3853 | Post-synaptic density protein 95 (PSD95) mRNA | U83192_at |
| 5869 | Surfacant Protein Sp-A1 Delta | HG3928-HT4198_s_at |
| 3856 | CUL-2 (cul-2) mRNA | U83410_at |
| 852 | Helix-Loop-Helix Protein Delta Max, Alt. Splice 1 | HG2525-HT2621_at |
| 5405 | ATM Ataxia telangiectasia mutated (includes | U33841_at |
| | complementation groups A, C and D) | |
| 5632 | LZTR-1 | D38496_s_at |
| 5299 | MXI1 mRNA | L07648_at |
| 29 | AFFX-PheX-3_at (endogenous control) | AFFX-PheX-3_at |
| 4878 | GB DEF = Transcriptional intermediary factor 2 | X97674_at |
| 4834 | Brca2 gene exon 2 (and joined coding region) | X95152_rna1_at |
| 6025 | FGFR4 Fibroblast growth factor receptor 4 | L03840_s_at |
| 2364 | LEUKOCYTE ELASTASE INHIBITOR | M93056_at |
| 3377 | IRF4 Interferon regulatory factor 4 | U52682_at |
| 3644 | GB DEF = 34 kDa mov34 isologue mRNA | U70735_at |
| 3803 | Basic-leucine zipper nuclear factor (JEM-1) | U79751_at |
| | mRNA | |
| 4986 | GB DEF = Flavin-containing monooxygenase 2 | Y09267_at |
| 5545 | PRPS1 Phosphoribosyl pyrophosphate synthetase 1 | X15331_s_at |
| 506 | Cysteine protease | D55696_at |
| 3009 | Pigment epithelium-derived factor gene | U29953_rna1_at |
| 3044 | Syntaxin 3 mRNA | U32315_at |
| 4095 | DNA polymerase alpha-subunit | X06745_at |
| 5224 | GB DEF = Axonemal dynein heavy chain | Z83805_at |
| | (partial, ID hdhc8) | |
| 5864 | Cell Division Cycle Protein 2-Related Protein | HG3914-HT4184_s_at |
| | Kinase (Pisslre) | |
| 6444 | LAMP2 Lysosome-associated membrane protein | S79873_s_at |
| | 2 (alternative products) | |
| 6475 | GARS Glycyl-tRNA synthetase | U09510_s_at |
| 238 | AMT Glycine cleavage system protein T | D14686_at |
| | (aminomethyltransferase) | |
| 1047 | Mac25 | HG987-HT987_at |
| 2354 | Ubiquitin carrier protein (E2-EPF) mRNA | M91670_at |
| 2570 | Clone A9A2BRB6 (CAC)n/(GTG)n repeat- | U00944_at |
| | containing mRNA | |
| 2951 | Protein associated with tumorigenic conversion | U25433_at |
| | (CATR1.3) mRNA | |
| 5495 | GB DEF = Ncx1 gene (exon 1) | X92368_at |
| 4070 | GNAI2 Guanine nucleotide binding protein (G | X04828_at |
| | protein), alpha inhibiting activity polypeptide 2 | |
| 1519 | GB DEF = (clone PEBP2aA1) core-binding | L40992_at |
| | factor, runt domain, alpha subunit 1 (CBFA1) | |
| | mRNA. 3′ end of cds | |

Preferable and Advantageous Result of the Data Mining System on a Case Study on B-CLL Leukaemia

The above described machine learning system is applied to the molecular genetic classification of B-CLL-patients based on five different experimental sources, which are previously published (Döhner et al. 2000, New England J Med. in press; Stratova et al. 2000, Intl. J. Cancer, in press):

1) Interphase FISH (fluorescence in situ hybridisation) analysis of clinically relevant chromosomal markers

2) Mutation analysis of a gene with diagnostic relevance

3) Gene expression profiling of ca. 1000 different genes

4) CGH (comparative genomic hybridisation) of B-CLL-patients

5) Clinical data base of B-CLL-patients

FIG. 3 describes the relationship between these experimental sources.

FISH Data Set Overview (n=325)

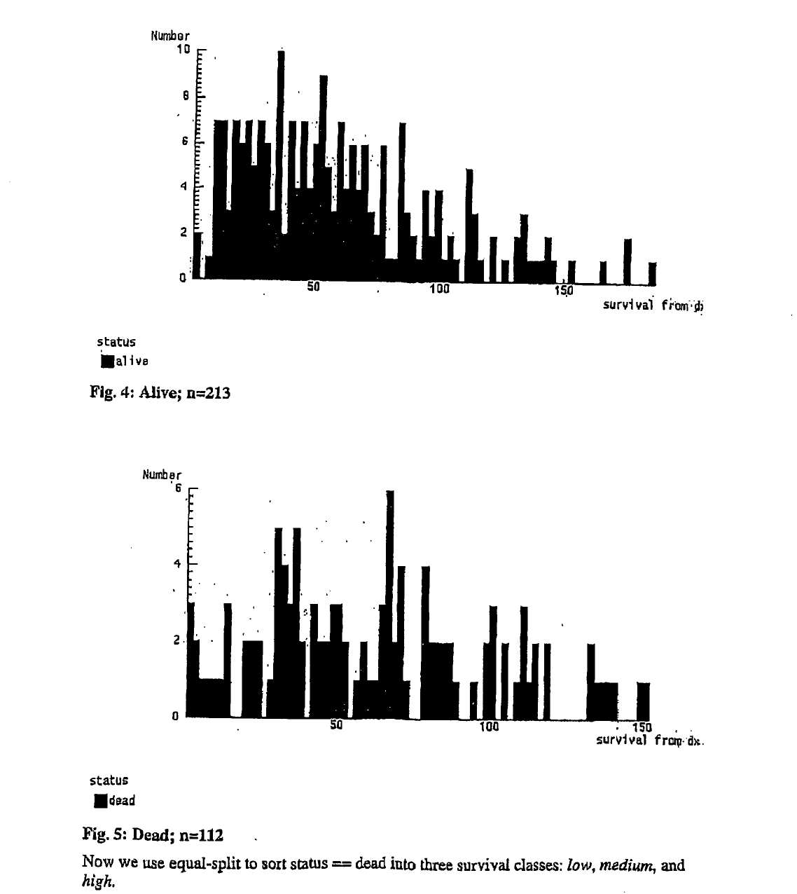

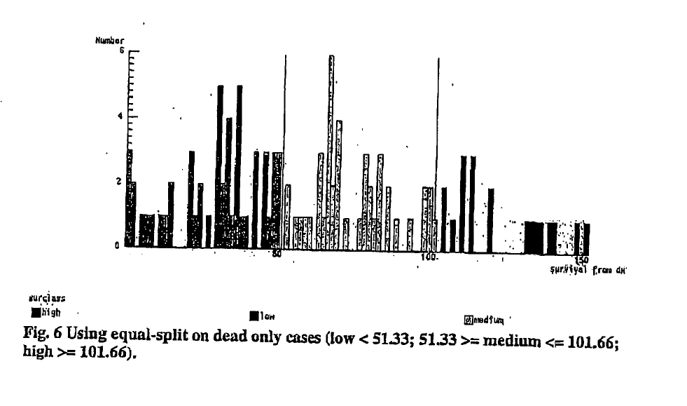

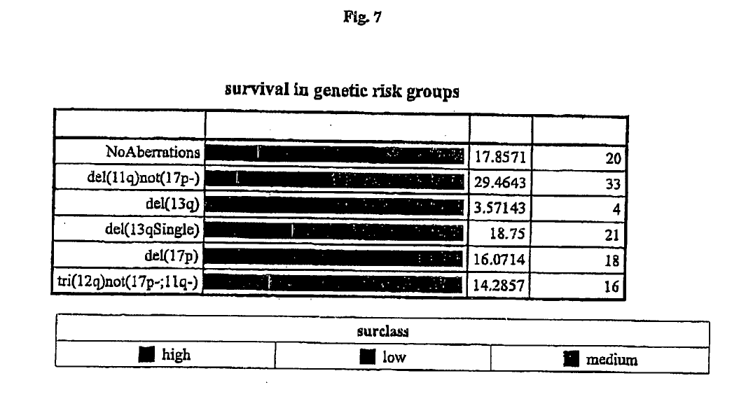

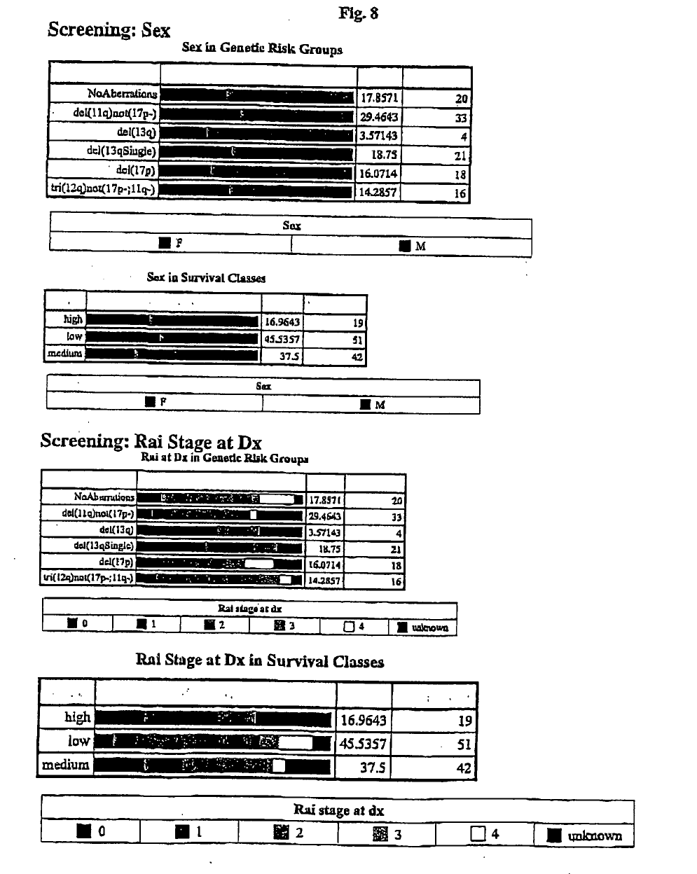

See FIGS. 4 to 7 for distribution of FISH on basis of status=dead/alive.

### Classification Using FISH Aberrations Only

Decision Tree

The decision tree confirms the main hypothesis/results of Doehner's,

Decision tree: predicted accuracy: tree=43.0%, rule set=43.0%. Special parameter settings: penalty=2.0 on missclassifying high as medium.

37p13 del (18.0, 0.833)->low

17p13 none

13q14 single del (21.0, 0.333)->high

13q14 single none

11q22-q23 del (33.0, 0.515)->medium

11q22-g23 none (40.0, 0.475)->low

Decision tree: predicted accuracy: tree=44.8%, rule set=45.7%. Special parameter settings: boosting fold=10. No special multiple models where obtain though.

Rule #1—estimated accuracy 53.6% (boost 53.6%]:

17p13 del (18.0, 0.833)->low

17p13 none

13q14 single del (21.0, 0.429)->medium

13q14 single none

11q22-q23 del (33.0, 0.515)->medium

11q22-q23 none (40.0, 0.475)->low

Neural Network

The neural net confirms the decision tree results and the Doehner hypothesis/results. At minimum a training accuracy of 58% was necessary to obtain consistent results.

| Input Layer: | 17 neurons |
|---|---|
| Hidden Layer #1: | 9 neurons |
| Hidden Layer #2: | 4 neurons |
| Output Layer: | 3 neurons |
| Predicted Accuracy: | 60.00% |

Relative Importance of Inputs

| | |
|---|---|
| 17p13: | 0.10489 |
| 13q14 single: | 0.07140 |
| 12q13: | 0.06054 |
| 11q22-q23: | 0.04223 |
| 13q14: | 0.04181 |
| 11q22-q23 single: | 0.02472 |
| normal y/n: | 0.01983 |
| 12q13 single: | 0.00785 |

### Association Using FISH Aberrations Only

From the two association analyses below, we can, by comparison, conclude that for the

high survival prognosis group: 13q14 single cm del is observed at least 3.68 more often than in the low group (there it is not observed above the threshold of >10%);

low survival prognosis group: 17p13 m del is observed at least 2.94 more often than in the high group (there it is not observed above the threshold of >10%);

and therefore 13q14 single==del seems to entail good survival prognosis whereas 17p13=del suggests a bad prognosis. This is consistent with the Doehner hypothesis/results.

Note, we observe a slightly higher of normal y/n==normal high group when compared to low. This is also consistent with the Doehner hypothesis/results.

Also, 11q22-q23==del is more pronounced 27.5%/21.1% in the low group. This is also consistent with the Doehner hypothesis/results.

surclass==high<=normal y/n==no (15:78.9%, 1.0)

surclass==high<=13q14==del (10:52.6%, 1.0)

surclass==high<=13q14 single==del (7:36.8X, 1.0)

surclass==high<=12q13==tri (5:26.3%, 1.0)

surclass==high<=11q22-q23==del (4:21.1%, 1.0)

surclass==high<=normal y/n yes (4:21.1%, 1.0)

surclass==high<=12q13 single==tri (2:10.5%, 1.0)

surclass==high<=6q21==del (2:10.5%, 1.0)

(“17p13 del” missing=>must be less than 10%)

surclass==low<=normal y/n==no (41:80.4%, 1.0)

surclass==low<=13q14 del (21:41.2%, 1.0)

surclass==low<=17p13=del (15:29.4%, 1.0)

surclass==low<=11q22-q23 del (14:27.5%, 1.0)

surclass==low<=normal y/n==yes (10:19.6%, 1.0)

surclass==low<=12q13 tri (7:13.7%, 1.0)

(“13q14 single” missing”=>must be less than 10%)

### Classification Using FISH Aberrations & Clinical Features

TABLE 1
Important Clinical Features

Clinical Feature
Sex
Rai stage at dx
albumin at study
abdom LN
hb at dx
Leucos at dx
LDH at dx
lymphadenopathy at dx
longest LN diameter at dx
Binet at dx

(see FIG. 8)

Screening: Binet Stage at Dx

FISH Aberrations & IgH Mutation over Risk Groups and Survival Classes (n=202) The underlying data set contains n=202 intersection of all 225 BCLL case and 202 IgH mutation data set: total n=202. The figures below depict the cases within the genetic risk and the survival classes in relation to IgH Mutations.

1. The relative proportion of IgH==yes in del(11q)not(17p-) is extremely low.

2. The relative proportion of IgH==yes in del(17p) and of IgH==yes del(6q;13q) is low.

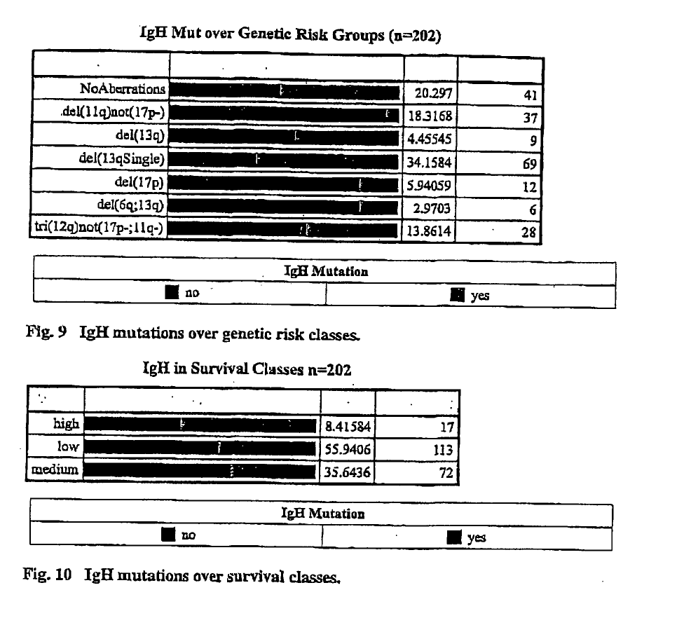

(see FIGS. 9 to 10)

Expression Against Genetic Risk Groups & Survival Classes

Potentially interesting genes: 1021, 472, 122, 1128, 833, 894, 1125, 138, 1299, 861, (see rule induction result below).

1. where high/low expression patterns of low(833) low(122), high(472), high(1125), high(138), high(1299), high(861) seem to be related to del(11q)not(17p);

2. where high/low expression patterns of low(894), low(833) del(13qSingle)

3. where high/low expression patterns of low(1021), high(1128) to del(17p)

All of these genes should individually be investigated against the genetic risk groups and in combination (as suggested above) against the genetic risk groups.

Gene Expression Patterns (n=325) gene 1021

1. A low expression pattern of gene 1021 occurs in ca. 4 out of 8 cases in del(17p) but not in the other three genetic risk groups. This is consistent with this a low expression pattern of that gene in ca. 5 of 22 in the low survival expectancy group when compared with zero occurrences in the other two survival classes.

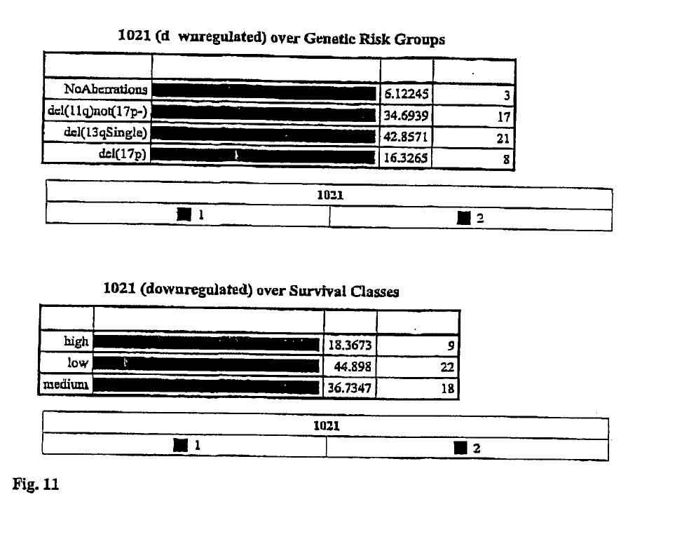

(see FIG. 11)

Gene Expression Patterns (n=325) gene 472 and 122.

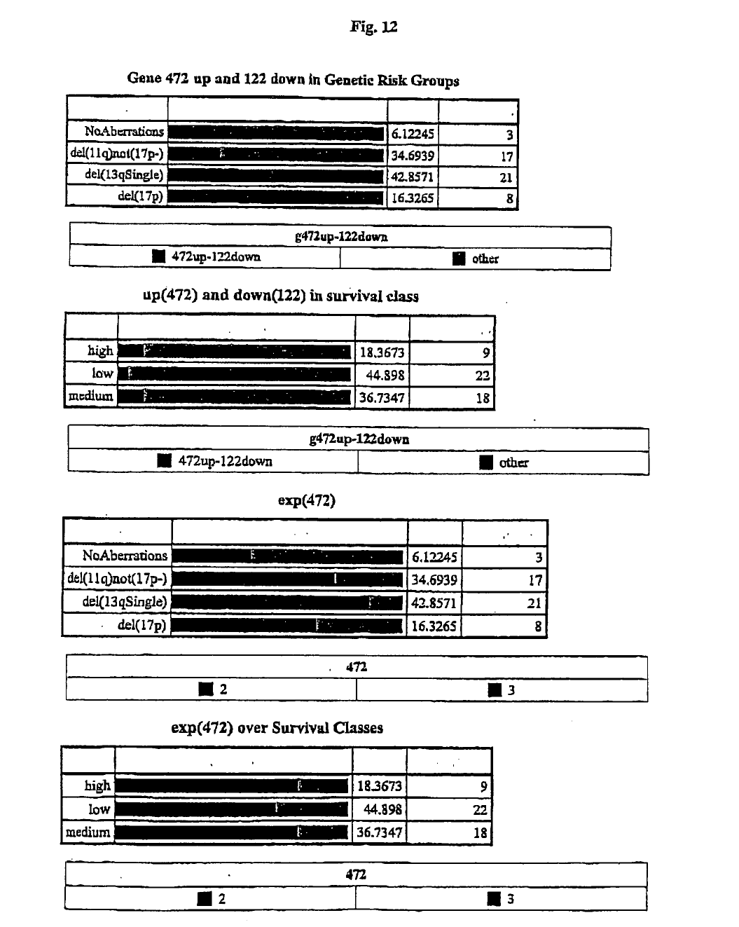

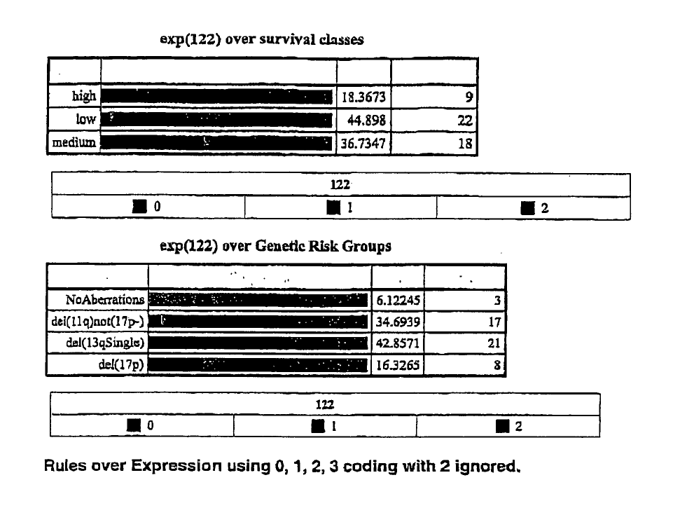

1. In the genetic risk group del(11q)not(17p-) we observe in 4 out of 17 (23.5%) cases a up-regulated 472 and a down-regulated. This pattern is not present in the other three genetic risk groups. The pattern up(472) and down(122) seems also be positive in terms of survival prognosis (see FIG. 12).

2. High expression levels of gene 472 are twice as often in del(17p) than in del(13qSingle), and they seem to be consistent with decreased survival prognosis (see FIG. 12).

3. The down regulation patterns of gene 122 are less strong. However, a clear gradient more frequent downregulation from del(17p9) to del(11q)bot(17p-) and low survival to high survival can be observed.

(see FIG. 12)

Rules Over Expression Using 0, 1, 2, 3 Coding with 2 Ignored.

```
Rules for NoAberrations:
    Rule #1 for NoAberrations:
        if 833 == 2
        then -> NoAberrations (3, 0.6)
Rules for del(11q)not(17p-):
    Rule #1 for del(11q)not(17p-):
        if 833 == 1
        and 894 == 2
        and 1128 == 2
        then -> del(11q)not(17p-) (5, 0.857)
    Rule #2 for del(11q)not(17p-):
        if 122 == 1
        and 472 == 3
        then -> del(11q)not(17p-) (4, 0.833)
    Rule #3 for del(11q)not(17p-):
        if 30 == 2
        and 1125 == 3
        then -> del(11q)not(17p-) (3, 0.8)
    Rule #4 for del(11q)not(17p-):
        if 30 == 2
        and 138 == 3
        then -> del(11q)not(17p-) (2, 0.75)
    Rule #5 for del(11q)not(17p-):
        if 30 == 2
        and 1299 == 3
        then -> del(11q)not(17p-) (4, 0.667)
    Rule #6 for del(11q)not(17p-):
        if 861 == 3
        and 1128 == 2
        then -> del(11q)not(17p-) (2, 0.5)
Rules for del(13qSingle):
    Rule #1 for del(13qSingle):
        if 138 == 2
        and 472 == 2
        and 861 == 2
        and 894 == 1
        and 1021 == 2
        and 1125 == 2
        and 1128 == 2
        and 1299 == 2
        then -> del(13qSingle) (16, 0.944)
    Rule #2 for del(13qSingle):
        if 122 == 2
        and 138 == 2
        and 861 == 2
        and 894 == 1
        and 1021 == 2
        and 1125 == 2
        and 1299 == 2
        then -> del(13qSingle) (13, 0.933)
    Rule #3 for del(13qSingle):
        if 833 == 1
        and 861 == 2
        and 1021 == 2
        and 1128 == 2
        then -> del(13qSingle) (37, 0.538)
Rules for del(17p):
    Rule #1 for del(17p):
        if 1021 == 1
        then -> del(17p) (3, 0.8)
    Rule #2 for del(17p):
        if 1128 == 3
        then -> del(17p) (4, 0.667)
Default : -> del(13qSingle)
```

Preferred Embodiment of a Molecular Classification of B-CLL-Patients by Bayesian Belief Networks

A Bayesian Belief Network was learned on data of 191 patients reconstructing the dependencies between chromosomal aberrations detected with FISH and presence/absence of IgH mutation. The structure of the network shows that some aberrations have no correlation with IgH Mutation status: 6q21, t(14q32), t(14;18), 12q13 as single aberration. The interesting paths in the network leading to the node IgH mutation thus implying the correlation of these facts are:

17p13=>IgHmutation,

11q22-q23=>IgHmutation,

12q13=>17p13=>IgHmutation,

13q14 single=>17p13=>IgHmutation

and others (red colored).

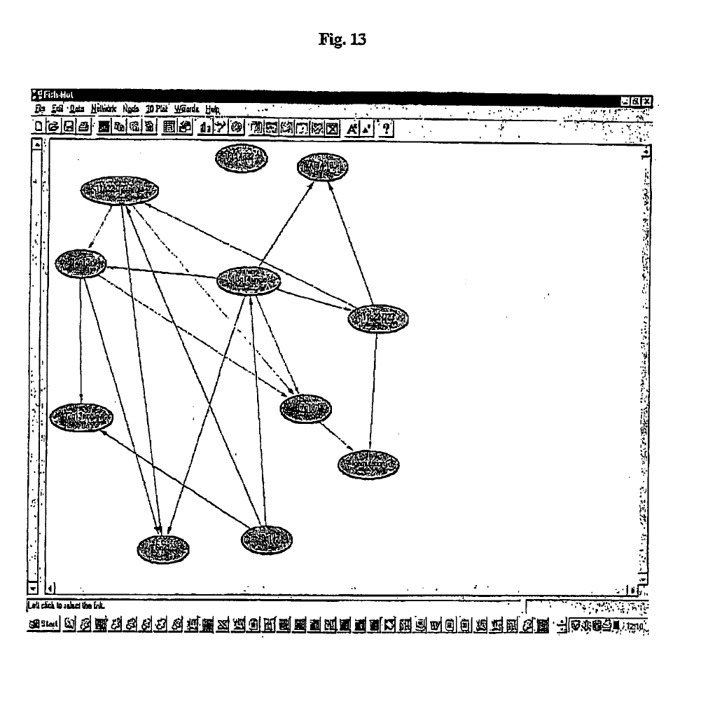

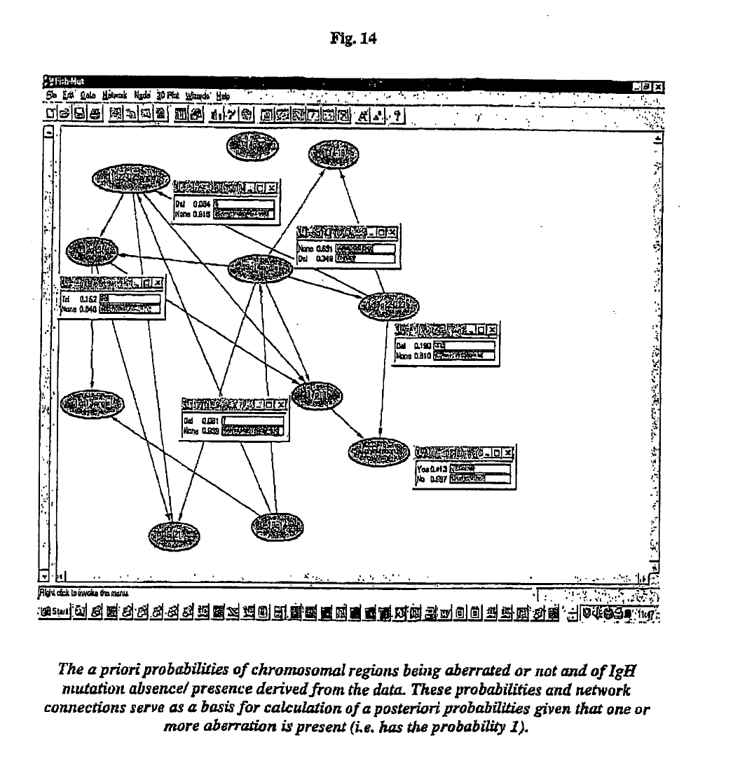

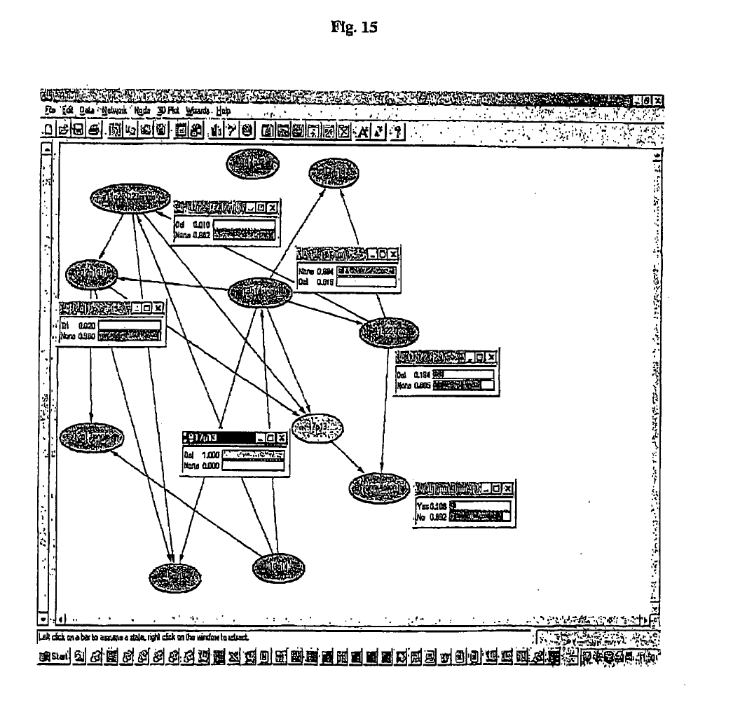

(see FIGS. 13 to 20)

Assuming that chromosomal region 17p13 is deleted with probability 1 we obtain that probability of no IgH mutt changes from 0.587 to 0.892 thus giving a clue that 17p13 deletion is strongly correlated with IgH mutation status no. (see FIG. 5)

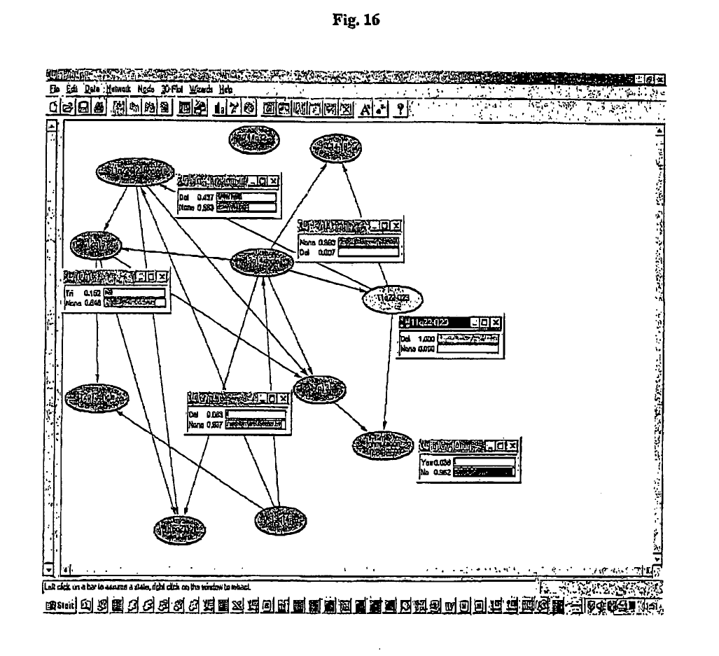

The deletion of the chromosomal region 11q22-q23 with probability 1 leads to changes of probabilities of all nodes on the directed path to the IgHmutation-node thus the probability of no IgH mutation changes from 0.587 to 0.962. (see FIG. 16)

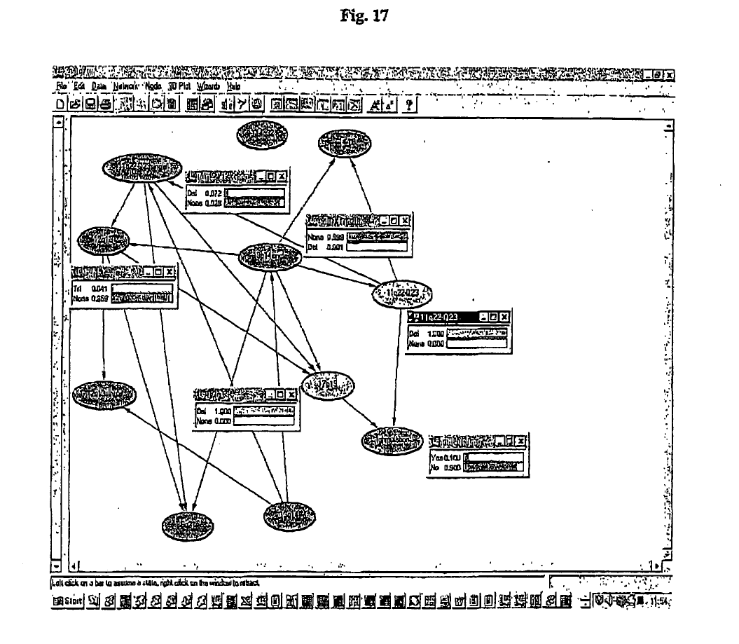

When the regions 11q22-q23 and 17p13 are both deleted with probability 1 the probability of no IgH mutation (0.900) becomes however less. (see FIG. 17)

When the chromosomal region 11q22-q23 is deleted but not the region 17p13 the probability of no IgH mutation becomes greater than the previous two probabilities—0.966, leading to hypothesis that 11q deletion (but not 17p deletion) is an independent category of abnormalities which correlate with IgH mutation status. (see FIG. 18)

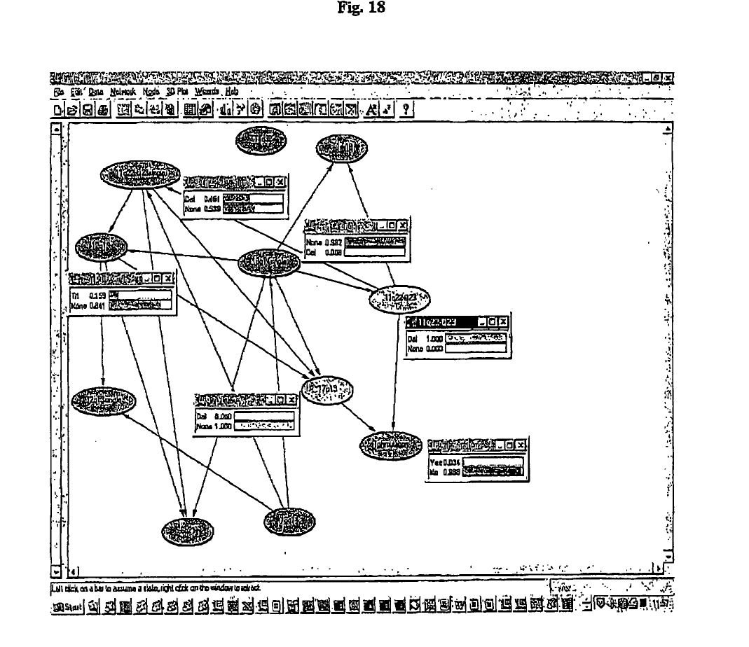

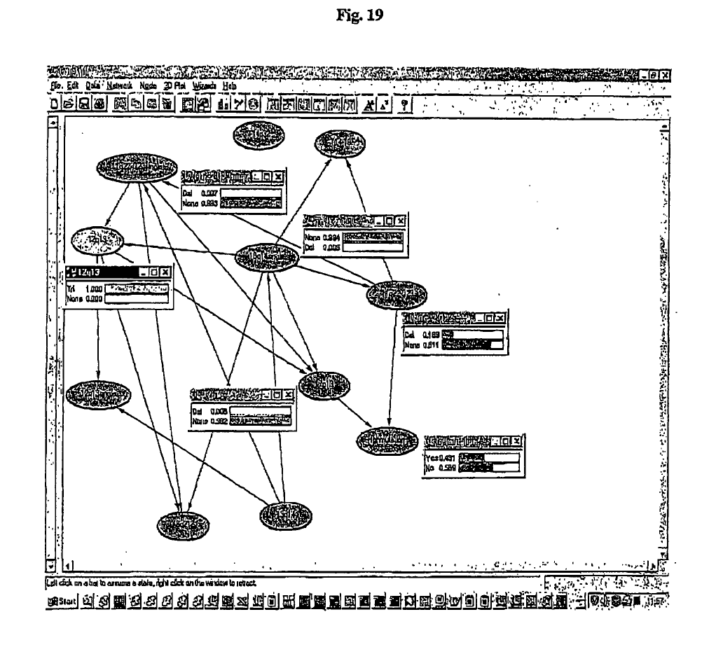

The trisomy of 12q13 region is connected with the presence of IgH mutation (its probability changes from 0,413 to 0,431). (see FIG. 19)

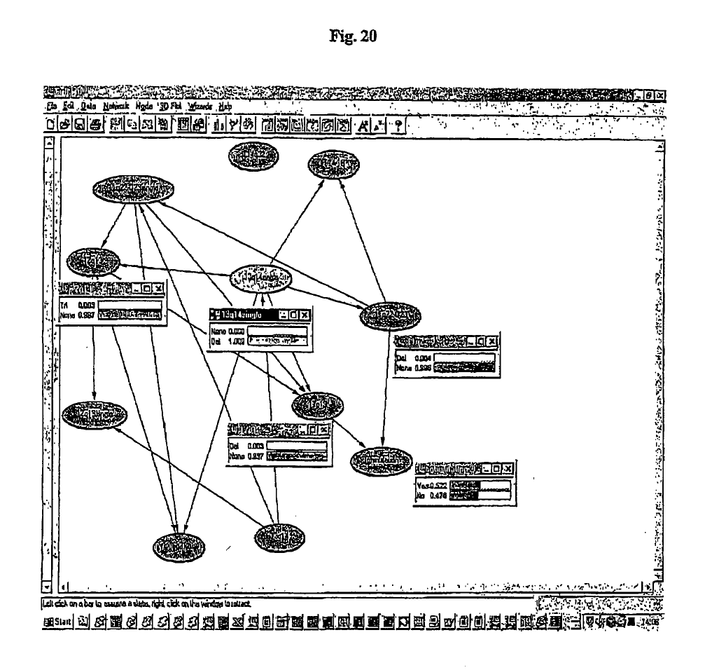

The deletion 13q14 as sole abnormality correlates positive with the presence of Igh mutation (probability change from 0,413 to 0,522). (see FIG. 20)

State-of-the-Art Methods Fail to Predict Genetical Risk Groups for B-CLL-Leukaemia Patients Based on Gene Expression Profiling

As outlined by the previous work by Stratova et al. (Intl. J. Cancer (2000), in press) no correlation between gene expression profiles and karyotype, which provides a genetic mask group classification, could be found. The following figures exemplify why the traditional method of testing the classification strength of genetic targets based on single gene expression levels fail to identify statistically relevant genetic targets, which are identified by our method (se below). The first figure shows that the Kaplan-Meyer-survival curves for patients with downregulated gene TGF-βR III (code no. 1021) are not significantly different as compared to patients with normal gene TGF-βR-III expression level within the same genetic risk group. Furthermore, only a tendency for statistical difference of Kaplan-Meyer-cures is found in comparison with all other patients in this study. However, no statistical difference can be found due to the small sample of patients included in his genewise comparison.

| | | | sur- | | |
|---|---|---|---|---|---|
| | | | vival | | |
| | | | from | | |
| key | status | cause of death | dx | 1021 | GeneticRiskGroups |
| 94PB153 | 1 | malignant disease | 15 | 2 | del(17p) |
| 95PB209 | 1 | malignant disease | 6 | 2 | del(17p) |
| 95PB88 | 1 | malignant disease | 36 | 1 | del(17p) |
| 95PB88 | 1 | malignant disease | 36 | 2 | del(17p) |
| 96PB72 | 1 | malignant disease | 50 | 2 | del(17p) |
| 96PB72 | 1 | malignant disease | 50 | 1 | del(17p) |
| 96PB925 | 1 | malignant disease | 28 | 1 | del(17p) |
| 97PB424 | 0 | | 2 | 2 | del(17p) |

N.B.:
Satus = 1 → dead; Status = 0 → alive
Discretization: ]0, 0.49] → downregulated → 1
[0.5, 2.00] → noise → 2

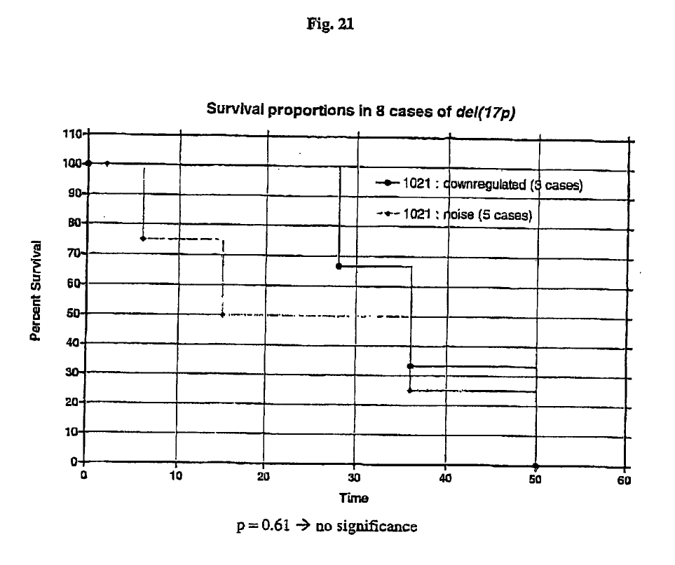

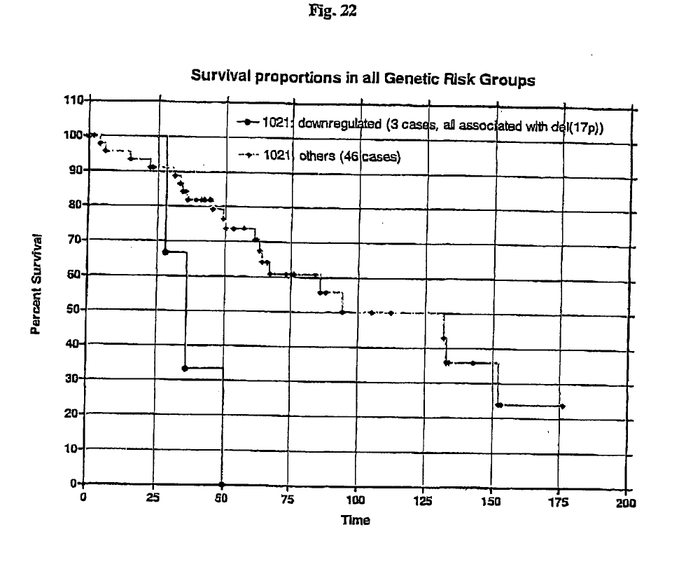

(see FIGS. 21 to 22)

| | Class 1 | Class 2 |
|---|---|---|
| Expression ratio | ]0, 0.49] | [0.50, 2.00] |
| Number of cases | 3 | 46 |
| Mean length of survival | 38.00 | 67.35 |
| [months] | | |
| Median survival [months] | 36.00 | 132.00 |
| Max. length of survival | 50 | 176 |
| [months] | | |
| Number of alive patients | 0 | 26 |

Molecular Genetic Results

Result of the Data Mining System on a Case Study on B-CLL Leukaemia Obtained by Proprietary Data Mining System

With the above described system it is possible to identify a set of genes (see figure below) which are able to classify the genetic risk of B-CLL leukaemia patients according to their gene expression profile. The factors below serve as potential genetic targets for new B-CLL-leukaemia drugs and therapy.

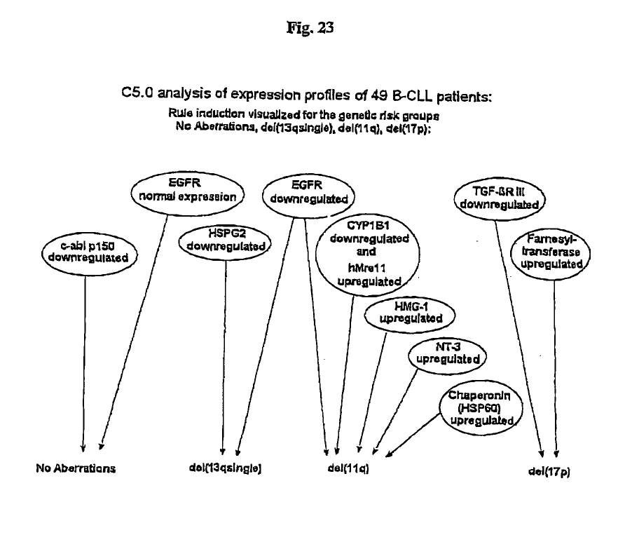

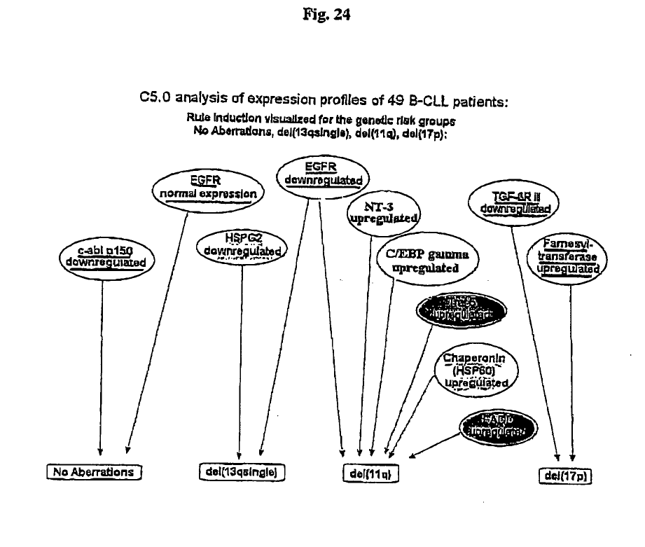

The figures show the genetic targets identified by the decision tree/rule induction method described above. In FIG. 1 the analysis was performed on the entire set of genes, whereas for FIG. 2 the analysis was performed only on non-redundant genes. (see FIGS. 23 to 24)

Another Preferred Embodiment of Molecular Classification of B-CLL-Patients by Data Mining

The original data set included expression profiles (real values) of 1559 human DNA probes of 47 patients with B-CLL analyzed with a microarray chip made by Incyte Pharmaceuticals, Inc. (USA) [5]. Based on fluorescence in situ hybridization (FISH) data for these patients and their correlation to survival time, four different genetic risk groups could be identified: (1) del(17p), (2) del(13qSingle), (3) del(11q), and (4) No aberrations [6]. Each patient has been assigned to one genetic risk group. Table 1 shows the number of patients in each group and the survival chances that are correlated with these groups:

TABLE 1
The number of patients per genetic risk group and the correlated
survival chances (fewer stars represent a lower survival chance).

| Genetic Risk | Number of | Survival |
|---|---|---|
| Group | patients | chances |
| del(13qSingle) | 21 | **** |
| No aberrations | 3 | *** |
| del(11q) | 17 | ** |
| del(17p) | 6 | * |

Before the data mining techniques were applied, the expression profiles are subject to a discretization step that produces three different symbolic values representing underexpressed, balanced, and overexpressed states. Furthermore, genes showing the same expression value in all 47 cases were excluded from further analysis, as they do not carry any discriminatory information with respect to the risk groups.

Basic Methodology

The basic analysis framework of this study is characterized by three distinct phases:

(1) data preprocessing: Remove control genes and discretize real values in underexpressed, balanced, and overexpressed states.

(2) discriminant analysis: Apply decision tree C5.0 to infer rules for the genetic risk groups.

(3) association analysis: Apply association algorithm to identify subsets of genes that are underexpressed, overexpressed, or balanced in the genetic risk groups.

Data Preprocessing

The gene expression profiles of the original data set are represented as absolute integral-numbered expression intensities. The decision tree algorithm used in this study is in principle able to handle continuous inputs. However, it is useful to distinguish between balanced expression, underexpression, and overexpression of genes. The cut-off levels of the expression profiles are not available, so that the gene expression profiles are discretized according to the following rules: (1) missing values are replaced by zero; (2) values greater than zero and smaller than (or equal to) 0.49 are considered as underexpressed, (3) values between 0.50 and 2.00 are considered as balanced, and (4) values greater than (or equal to) 2.01 are considered as overexpressed.

The choice of these cut-off levels is based on a visual inspection of the distribution of the expression profiles. FIG. 24 depicts the discretization.

For all data preprocessing operations, proprietary algorithms, implemented with MATLAB 5.3 [7], have been used.

Classification

Decision Tree Algorithm

Decision trees are preferably used for classification and prediction tasks and follow a kind of top-down, divide-and-conquer learning process. The working scheme of a decision tree algorithm can be described in the following way. The attribute that based on an information gain measure—provides the best split of the cases with respect to the attribute to be predicted is selected as the root node of the tree. A branch for each possible value of the tree is generated from this root node, splitting the data set into subgroups. These steps are recursively repeated for each of the branches with only those cases that reach the respective branch. The algorithm stops the processing of a certain branch when all associated members were classified equally. These end nodes of a branch are hence called leaf nodes. The root node of a decision tree is regarded as the most important attribute with respect to the classification task. The importance of the following nodes is sequentially decreasing. Due to this, decision trees are capable of extracting rules by which the classification was achieved. In contrast to other widely used classification algorithms (e.g., artificial neural networks), these rules are understandable for humans.

The decision tree algorithm used in the presented study is the powerful SPSS' Clementine [8] implementation of Ross Quinlan's C5.0 [9], the advanced successor of the well known C4.5 [10]. One of the major advantages of C5.0 is its capability to generate trees with a varying number of branches per node unlike other decision tree algorithms like CART that provide binary splits [11]. In order to improve the accuracy of a classifier, Clementine's C5.0 implements a cross-validation method called boosting [12]. This method maintains a distribution of weights over the data set, where initially each case is assigned the same weight. Those cases that were misclassified in the first classification process get a higher weight and the data set is classified again. This provides an accentuation of the hard-to-classify cases resulting in (1) an elevated accuracy of the classifier and (2) more than one rule set that denotes the classifier.

Classification Results

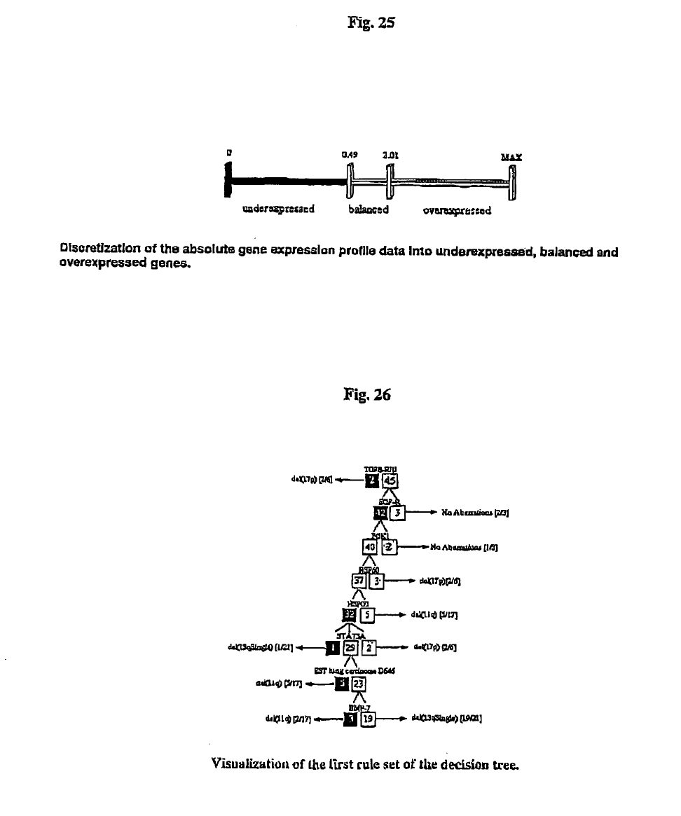

Applying C5.0 to the data set of 47 patients with B-CLL was performed with the task to predict the genetic risk group of each individual case. The estimated accuracy using 3 fold boosting was 100% meaning that with a model made up of these 3 rule sets, it was possible to predict each case within the data set correctly. The extracted rule sets identified a number of genes the algorithm recognized as important for the classification into the four genetic risk groups. The result of the first rule set has been visualized in FIG. 25.

Presented is the first rule set of 3 comprising the prediction model. White boxes indicate a balanced gene expression state, black boxes underexpressed, and grey boxes overexpressed states, respectively, Abbreviations of genes are written on top of the respective boxes (TFGβ-RIII: transforming growth factor receptor type III; EGF-R: epidermal growth factor receptor; PGK-1: phosphoglycerate kinase 1; HSP60: chaperonin, HSPG2: heparansulfate proteoglycan; Stat5A: signal transducer and activator of transcription 5A; EST: estimated sequence tag; BMP-7: bone morphogenic protein 7). Numbers inside the boxes represent the number of cases that follow this rule. The numbers in brackets written behind the genetic risk groups include the number of cases of the respective group that follow this rule and the total number of cases within this group. The rule set in FIG. 26 has to be read as follows. The root node TGFβ-RIII splits into balanced expression status of the gene counting 45 of the 47 cases in the whole data set (white box). The second split refers to the underexpressed status that holds 2 cases (black box). The first rule classifies 2 of the 6 cases of group del(17p) into this group and there is no other case where this rule applies in the whole data set of those cases where TGFβ-RIII is balanced, EGF-R is underexpressed in 42 cases and balanced in 3 cases, 2 of these 3 cases are covered by the rule “if TGFβ-RIII is balanced and EGF-R is balanced then classify to group “No aberrations” which resemble 2 of all 3 cases in this genetic risk group. Thus, this very rule describes one additional case that does not belong to the group No aberrations but to another (which is del(11q)). Interestingly, 19 out of the 21 cases (90%) comprising the group del(13qSingle) are characterized by one rule with the root node TGFβ-RIII balanced and ending at the leaf node BMP-7 balanced. The group del(13qSingle) is known to be the best with respect to the survival chances. FIG. 26 depicts a Kaplan-Meyer survival analysis of these 19 patients vs. all other patients.

Every rule has to be lead from the root node to its respective leaf node. Whenever the number in a box with an arrow pointing towards a genetic risk group is equal to the first number in brackets listed after the respective group the corresponding rule applies only to cases of this group. Furthermore, with the exception of 4 cases belonging to group del(11q), every case is classified with the presented rule set. The remaining cases can be classified taking all three rule sets of the decision tree model together (data not shown).

As it is common in gene expression data sets the number of cases (in our study 47) is by for too low with respect to the attributes considered. Thus it was not suitable to split the data set into a training and a test set to which the model could have been applied in order to evaluate the strength of the rules learned from the training data. To address this limitation, we performed a 20 fold cross-validation, that divided the data set into 20 equally sized blocks according to the distribution of the cases whereby holding out a number of cases for testing, Thereafter a classifier was built upon each of the 20 reduced sets, and it was tested on the respective hold-out set. The cross-validation yielded a test accuracy of 40% (with a standard error of 6.8%).

The biological implications of decision tree results are non-trivial to interpret. On the one hand, you have to look at each of the genes that were found to be important to distinguish between the given groups. Table 2 gives a summary of genes in the three rule sets provided by C5.0. On the other hand, the genes highlighted by the classification algorithm can be seen on a more systemic view in context of the pathways they are involved in. An overlap of some pathways can be seen, e.g. genes encoding for EGF-R, GRB-2, and MAP2K2 are listed in Table 2. It has been shown that GRB-2 associates with EGF-R, and both gene products are entangled in the RAS-pathway, as is MAP2K2. Thus it is tempting to speculate whether the mentioned pathways do play a concerted role in B-CLL, which, of course, has to be recognized by molecular biological experiments. This demonstrates the power of applying machine learning techniques to complex data sets so far, as the results formulate hypotheses that have to be validated by biological means.

TABLE 2
Gene abbreviations, gene accession
numbers (Access#), and keywords of
biological role of genes found by the
decision tree algorithm (PDGF-R: platelet
derived growth factor receptor; n.p.: not provided).

| Gene | Access# | Biological keywords |
|---|---|---|
| TGFβ-RIII | L07594 | Apoptosis |
| EGF-R | U48722 | Apoptosis |
| PGK-1 | n.p. | Glycolysis |
| HSP60 | M34664 | Stress factor |
| HSPG2 | M85289 | Stress factor |
| Stat5A | U43185 | JAK/Stat pathway |
| BMP 7 | X51801 | Growth factor |
| AK2A | U39945 | essential for maintenance and |
| | | cell growth |
| PAFAH | n.p. | inactivates platelet-activating |
| | | factor |
| bcl-2 | M13994 | Apoptosis regulator |
| PPP5 | X89416 | RNA biogenesis? |
| HIAP2/BIR | U45879 | Apoptotic suppressor |
| C2 | | |
| GRB-2 | L29511 | EGF-R/PDGF-R pathway |
| MCP- | n.p. | Chemotactic factor/augments |
| 1/SCYA2 | | monocyte anti-tumor activity |
| PDHA1 | J03503 | Pyruvate metabolism |
| PLAUR | U08839 | mediates the signal |
| | | transduction activation effects |
| | | of urokinase plasmin |
| MAP2K2 | U12779 | Ras/Raf pathway |
| IGFBP4 | U20982 | Enhancer of apoptosis |

In summary, Table 2 presents genes known to be involved in apoptosis, stress reaction, metabolism, and tumor relevant pathways despite a few not correlated to any of these categories. In addition to the study of Stratowa et al. [5] that found genes involved in lymphocyte trafficking to be of prognostic relevance in B-CLL patients using the same gene expression data set, the majority of the genes found in our study are located in tumor relevant pathways.

In conclusion, the consequences arising from the fact that the studied data set comprised only 47 patients have to lead to additional investigations with a higher number of patients involved. This would facilitate the learning process of the algorithm, and the model could be tested with unseen data. On the other band, it can be hypothesized that those genes found by the decision tree algorithm may play a pivotal role in B-CLL.

Association

Maximum Association Algorithm

The goal of mining association rules in a data space is to derive multi-feature correlations between the attributes. Association algorithms associate a particular conclusion with a set of conditions. In commercial applications, association rules can be used to determine what items are often purchased together by customers, and use that information to arrange, e.g., store layout. A typical rule in this domain is given by the following expression: “80% of the customers that purchase product X also purchase product Y.” Association rules differ from classification rules in that they can be used to predict any attribute and not just a class [13]. Furthermore, classification rules are intended to be used as a set Association rules, on the other hand, express different intrinsic regularities in the data set, so that they can be used separately. The two most important measures of interest for association rules are the coverage (also called support) and the accuracy (also called confidence). The coverage of an association rule is the number of cases in which it is applicable (i.e. in which the antecedent—the if-clause—of the rule holds). The accuracy is the number of cases that the rule predicts correctly, expressed as a proportion of all cases it applies to (i.e. the number of cases in which the rule is correct relative to the number of cases in which it is applicable). Table 3 shows an example for association rules in a gene expression data set:

TABLE 3
An example for association rules
in a gene expression data set.

| Patient ID | Genetic Risk Group | Gene_X | Gene_Y | Gene_Z |
|---|---|---|---|---|
| 1 | A | 1 | 1 | 1 |
| 2 | A | 1 | 1 | −1 |
| 3 | A | 0 | 1 | 0 |
| 4 | B | 1 | 1 | 0 |
| 5 | B | −1 | 0 | 0 |

One association rule that can be derived from this data set is given by the following expression:

if Gene_X=1 and Gene_Y=1 then Genetic Risk Group=A (coverage: 3 (0.6), accuracy: 2/3).

The if-clause of the rule applies three times, for the case #1, #2, and #4. Therefore, the coverage is 3 (or, relative to the number of all cases of the data set, 0.6). For case #1 and #2, the then-clause is correct, but for case #4, it is not. Consequently, the accuracy is 2/3. This example clearly illustrates that even from a tiny data set, a huge amount of association rules can be derived. Therefore, only the “most interesting” rules, based on their coverage and accuracy, should be capitalized.

In our analysis, we were not mainly interested such association rules, but rather in associations of genes that have different expression states in the different genetic risk groups. For the gene expression data set, such an association could consist of the following statement: “In the genetic risk group del(17p), Gene_X, Gene_Y and Gene_are underexpressed in 100% of the cases, but in the group del(13qSingle), they are overexpressed in 100% of the cases.” If a gene is over- or underexpressed in 100% of the cases of a genetic risk group A, we call this gene “totally overexpressed in A”, respectively “totally underexpressed in A”.

The advantage of association rule algorithm over decision tree algorithms is that associations can exist between any of the attributes. A decision tree algorithm will only build rules with a single conclusion, whereas association algorithms attempt to find many rules, each with a different conclusion. On the other hand, associations may exist between a plethora of attributes, so that the search space for association algorithms can be very large. Therefore, association algorithms can require orders of magnitude more time to run than a decision tree algorithm. The Apriori algorithm [14], e.g., cannot reveal all possible associations because of the complexity of the search space. Therefore, we developed an alternative algorithm, called the maximum association algorithm, that is able to reveal all sets of associations that apply for 100% of the cases in one genetic risk group. This algorithm operates in four steps, each of them yielding interesting results.

In the first step, the algorithm screens the matrix of discretized expression data and identifies those genes that are either totally under- or totally overexpressed in one specific genetic risk group. To achieve this, the algorithm slides a window over all genes and all genetic risk groups. The following figure illustrates the procedure for the group del(13qSingle) and the gene #1. (Note that this is only a simplified example to illustrate the concept of the algorithm; the expression values in this example do not correspond to the real values in the data set of this study.) (see FIG. 27)

The sets of under- or overexpressed genes of one group are of course not necessarily disjoint with the sets of another group, for a specific gene can be underexpressed for all patients of a genetic risk group A and also for all patients of a group B.

The results of the first step of the maximum association algorithm have been stored in a cytogenetics database that has been developed for data mining purposes [15]. Via user-friendly graphical interfaces, a remote access to these results is possible, and even complex queries can be easily formulated. One example for such a query is the following: “Select all genes that are totally overexpressed in the genetic risk group del(17p), totally underexpressed in the group del(13qSingle), and neither totally expressed in No aberrations nor in del(11q).

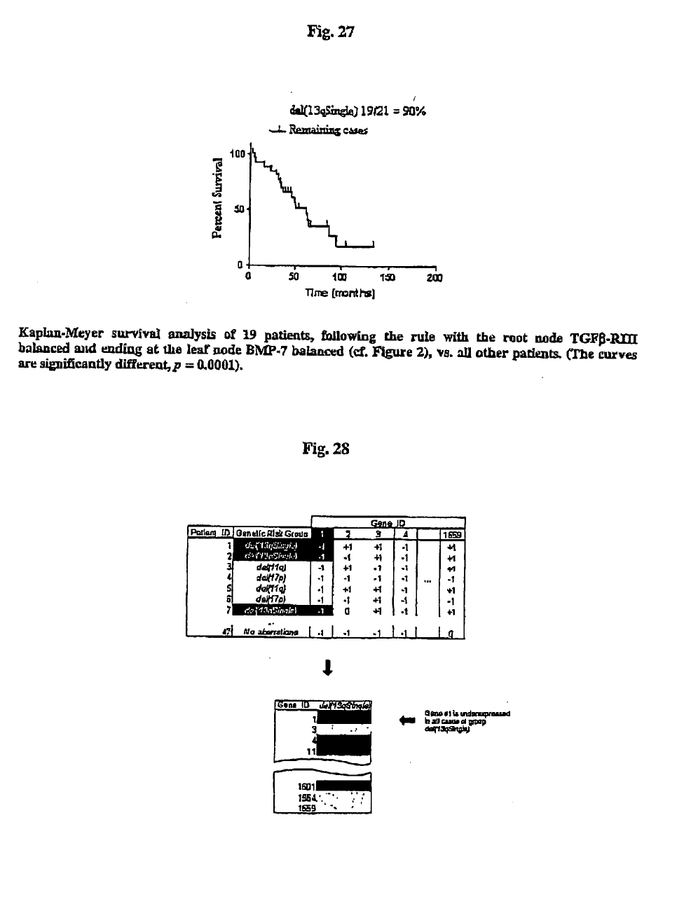

In the second step, the algorithm eliminates those genes that are equally expressed in all genetic risk groups. If a specific gene is equally expressed in all groups, it has no discriminatory functions and hence it is removed. FIG. 5 illustrates the elimination process. The arrows indicate which genes will be removed; here, gene #1, #4, #6, and #1555 will be excluded from further analysis. (see FIG. 28)

In the third step, the algorithm operates as follows: if a specific gene is totally under or totally overexpressed in a genetic risk group A but not in a group B, then the algorithm counts the number of cases in B for which this gene is balanced, the number of cases for which it is underexpressed, and the number of cases for which it is overexpressed. The expression state of this gene for the group B is then determined based on a majority vote: (1) if the number of cases for which this gene is underexpressed exceeds both the number of cases where the same gene is overexpressed and the number of cases where this gene is balanced then this gene will be regarded as underexpressed by the majority; (2) if the number of cases for which this gene is overexpressed exceeds both the number of cases where the same gene is underexpressed and the number of cases where this gene is balanced, then this gene will be regarded as overexpressed by the majority; (3) if this gene is balanced in at least 50% of the cases, then it will be regarded as balanced by the majority.

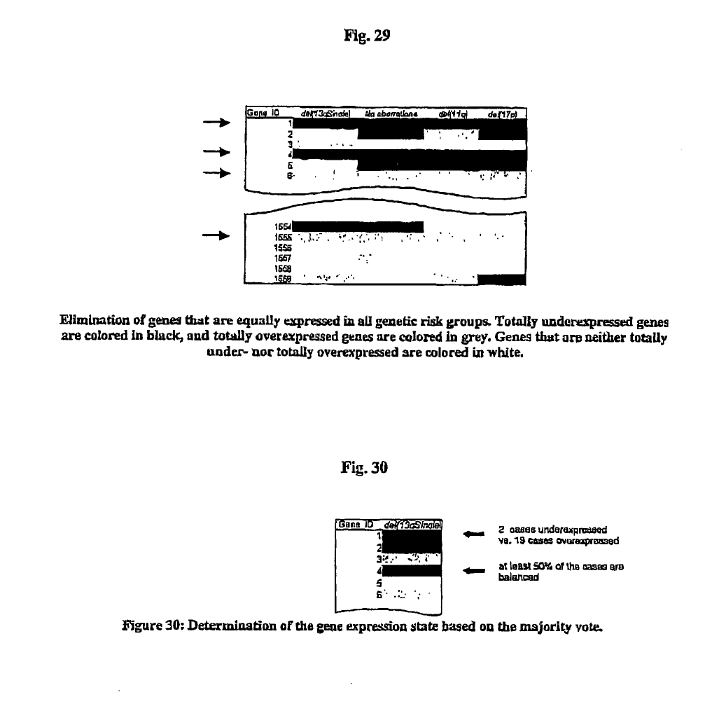

(see FIG. 29)

For example, let gene #2 be underexpressed for 2 cases of the group del(13qSingle), and let this gene be overexpressed in the remaining 19 cases. Then for this group, gene #2 will be regarded as overexpressed by the majority. FIG. 30 illustrates this operation:

After the operation in the third step, some genes can be equally expressed in all genetic risk groups. These genes are removed in the fourth step. This procedure is analogous to the operation described in the second step.

The maximum association algorithm has been developed with MATLAB 5.3 [7]. Although the analysis has been carried out on a standard PC, the algorithm could be executed in a very reasonable time.

[image]

In total, 14 genes “survived” the selective operations of the maximum association algorithm. The two most interesting genes are highlighted in Table 4. In the genetic risk groups del(17p) and in the group No aberrations, the gene with the accession number J03202 is totally overexpressed, whereas it is overexpressed by the majority in the group del(13qSingle) and balanced by the majority in del(11q). The gene identified by the accession number M31303 is totally underexpressed in the group del(17p), while it is balanced by the majority in all other groups,

Discussion

When the number of features exceeds the number of observed cases, decision trees are prone to overfitting, i.e. the decision tree tends to encode the idiosyncrasies of the specific data set instead of inferring generalized rules. In this study, the number of attributes (1559 human DNA probes) exceeds by for the number of cases (47 patients). Consequently, it was not possible to improve the decision tree's ability to generalize by splitting the data set into a training set and a rest set. Therefore, we decided to perform a 20 fold cross-validation, that divided the data set into 20 equally sized blocks. In each cross-validation fold, a number of cases have been hold out for training, and another number of cases for testing. In the first cross-validation fold, each case had the same probability to fall into the training set or the test set. To those cases that have been misclassified in the n-th cross-validation fold was assigned a higher probability to fall into the training set of the (n+1)-th fold. Ibis procedure called boosting provides an accentuation of the hard-to-classify cases and results in a more precise and reliable classifier. The resulting model is fully satisfactory with a test accuracy of 40% (standard deviation of 6.8%.).

Intelligent data analysis and data mining methods are extremely important for the present and future developments of systems biology. Molecular biologists are currently engaged in some of the most impressive data collection projects, for example, genome sequencing, gene expression profiling, and protein interaction analysis. These project are generating an enormous amount of data related to structure, function, behaviour, and control of biological systems. The analysis and interpretation of this wealth of data will deeply affect and improve our understanding of biological systems and their underlying mechanisms. However, the elicitation and the representation of biological knowledge are extremely challenging tasks, which are demanding powerful and sophisticated data mining methodologies. Most widely used data mining software do not address the specific requirements of life science applications. On the other hand, the new association algorithm presented in this paper has been tailored for association mining in large data sets of gene expression data where even sophisticated methods like the Apriori algorithm would fail due to the complexity of the data.

### REFERENCES

[1] Kohonen T. Self-organized formation of topologically correct feature maps. Biol Cybern, 43:59-69, 1982.

[2] Granzow M., Berrar D., Dubitzky W., Schuster A., Azuaje F. J., Eils, R. Tumor Classification by Gene Expression Profiling: Comparison and Validation to Five Clustering Methods. ACM SIGBIO Newsletter, vol. 21, no. 1: 16-22, April 2001.

[3] Zwiebel J. A, Cheson B. D. Chronic lymphocytic leukemia: staging and prognostic factors, Semin. Oncol. 25, 42-59 (1998).

[4] Julius G., Merup M. Cytogenetics in chronic lymphocyte leukemia. Semin. Oncol. 25, 19-26 (1998).

[5] Stratowa C., Löffler G., Lichter P., Stilgenbauer S., Haberl P., Schweifer N., Döhner H., Wilgenbus, K. K. cDNA Microarray gene expression analysis of B-cell chronic lymphocytic leukemia proposes potential new prognostic markers involved in lymphocyte trafficking. J Cancer 91: 474-480, 2001.

[6] Döhner H., Stilgenbauer S., Benner A., Leupolt E., Krober A, Bullinger L., Döhner K, Bentz M., Lichter P. Genomic aberrations and survival in chronic lymphocytic leukemia. N Engl J Med December 28, 2000;343(26);1910-6.

[7] Mathworks MATLAB http://www.mathworks.com/.

[8] SPSS Clementine. http://www.spss.com/clementine.

[9] RuleQuest Research Data Mining Tools. http://www.rulequest.com

[10] Quinlan J. R. C4.5: Programs for machine learning. Morgan Kandar, San Francisco, 1993.

[11] Bery M. J., Linoff G. Data Mining Techniques For Marketing, Sales and Customer Support, John Wiley & Sons, Inc., New York, 1997.

[12] Freund Y., Schapire R. E. A decision-theoretic generalization of online learning and an application to boosting. Journal of Computer and System Science, 55(1): 119-139; 1997]

[13] Witten I. H., Frank E. Data Mining: Practical Machine Learning Tools and Techniques with Java Implementations, Morgan Kaufmann Pub. San Francisco, 1999.

[14] Agrawal R., Ramakrishnan S. Fast Algorithms for Mining Association Rules. Proc, 20th Int. Conf. Very Large Data Bases, VLDB, 1995.

[15] Berrar D., Dubitzky W., Solinas-Toldo S., Bulashevska S., Granzow M., Conrad C, Kalla K., Lichter P., Eils R. A Database for Comparative Genomic Hybridization Analysis. IEEE Eng Med Biol Mag. 20(4): 75-83, 2001.

1. Method for classifying genetic conditions, diseases, tumors etc., and/or for predicting genetic diseases, and/or for associating molecular genetic parameters with clinical parameters and/or for identifying tumors by gene expression profiles etc., the method having the following steps:
(a) providing molecular genetic data and/or clinical data,
(b) optionally automatically generating classification, prediction, association and/or identification data by means of machine learning, and
(c) automatically generating (further) classification, prediction, association and/or identification data by means of supervised machine learning.

2. Method according to claim 1, wherein for step (a) molecular genetic data and clinical data are provided.

3. Method according to claim 1 or 2, wherein the machine learning system is an artificial neural network learning system (ANN), a decision tree/rule induction system and/or a Bayesian Belief Network.

4. Method according to any one of the preceding claims, wherein for generating the data in the machine learning system at least one decision tree/rule induction algorithm is used.

5. Method according to any one of the preceding claims, wherein the data automatically generated is tumor identification data making use of gene expression profiles and being generated by a clustering system wherein further the clustering system makes use of one or more of the following clustering methods: Fuzzy Kohonen Networks, Growing cell structures (GCS), K-means clustering and/or Fuzzy e-means clustering.

6. A Method according to any one of the preceding claims, wherein the data automatically generated is tumor classification data being generated by Rough Set Theory and/or Boolean reasoning.

7. Method according to any one of the preceding claims, wherein for automatically generating the data use is made of FISH, CGH and/or gene mutation analysis techniques.

8. A. Method according to any one of the preceding claims, wherein before step (a) data is collected by means of gene expression techniques, preferably by cDNA microarrays, and then analyzed for providing the molecular genetic data.

9. Method according to any one of the preceding claims, with one or more algorithm(s) as specified in the description.

10. Computer program comprising program code means for performing the method of any one of the preceding claims when the program is run on a computer.

11. Computer program product comprising program code means stored on a computer readable medium for performing the method of any one of claims 1-10 when said program product is run on a computer.

12. Computer system, particularly for performing the method of any one of the claims 1-9 comprising:
(a) means for providing molecular genetic data and/or clinical data,
(b) optional means for automatically generating classification, prediction, association and/or identification data by means of a machine learning system, and
(c) means for automatically generating (further) classification, prediction, association and/or identification data by means of a supervising machine leaning system.

13. Computer system according to claim 12, wherein the system comprises means for carrying out the method steps as recited in one or more of claims 1 to 9.

14. Use of a data mining system according to the description and/or the method according to any one of claims 1-9.

15. Use of a method according to any one of claims 1-9 for classifying genetic conditions, diseases, tumors etc., and/or for predicting genetic diseases, and/or for associating molecular genetic parameters with clinical parameters and/or for identifying tumors by gene expression profiles etc.

16. Data, genes and/or genetic targets etc., obtainable by a method according to any one of claims 1-9, a computer program according to claims 10 or 11, a computer system according to claims 12 or 13, a use according to claims 14 or 15 and/or by any other way as described or implied by the specification.

17. Method for the production of a diagnostic composition comprising the steps of the method according to any one of claims 1-9 and the further step of preparing a diagnostically effective device and/or collection of genes based on the results obtained by the method of any one of claims 1-9.

18. Use of a gene or a collection of genes for the preparation of a diagnostic composition for classifying genetic diseases, tumors etc., and/or for predicting genetic diseases, and/or for associating molecular genetic parameters with clinical parameters and/or for identifying tumors by gene expression profiles etc.

19. Method for determining a treatment plan for an individual having a disease, such as cancer, with the following steps:
obtaining a sample from the individual,
deriving individual molecular genetic data and/or clinical data from the sample,
using a classifying method according to any one of claims 1-9,
comparing the individual molecular genetic data and/or clinical data from the sample with the classification obtained by the classifying method and
determining a treatment plan according to the classification result.

20. Method for diagnosing or aiding in the diagnosis of an individual with the following steps:
obtaining a sample from the individual,
deriving individual molecular genetic data and/or clinical data from the sample,
using a classifying method according to any one of claims 1-9,
comparing the individual molecular genetic data and/or clinical data from the sample with the classification obtained by the classifying method,
determining a treatment plan according to the classification result and
diagnosing or aiding in the diagnosis of the individual.

21. Method for determining a drug target of a condition or disease of interest with the following steps:
obtaining a classification with a method according to any one of claims 1 to 9 and
determining genes that are relevant for the classification of a class.

22. Method for determining the efficiency of a drug designed to treat a disease class with the following steps:
obtaining a sample from an individual having the disease class,
subjecting the sample to the drug,
classifying the drug exposed sample with a method according to any one of claims 1 to 9.

23. Method for determining the phenotypic class of an individual with the following steps:
obtaining a sample from the individual,
deriving individual molecular genetic data and/or clinical data from the sample,
establishing a model for determining the phenotypic classes with a method according to any one of claims 1 to 9, and
comparing the individual data with the model.
